(19) United States
(12) Patent Application Publication
(10) Pub. No.: US 2022/0260596 A1
(43) Pub. Date: Aug. 18, 2022

(54) COMPOSITIONS AND METHODS FOR BLOOD ANEMIA DETECTION

(71) Applicant: CASE WESTERN RESERVE UNIVERSITY, Cleveland, OH (US)

(72) Inventors: Umut Gurkan, Cleveland, OH (US); Ran An, Cleveland, OH (US)

(21) Appl. No.: 17/622,062

(22) PCT Filed: Jun. 29, 2020
(86) PCT No.: PCT/US2020/040165

§ 371 Date: Dec. 22, 2021

Related U.S. Application Data
(60) Provisional application No. 62/867,318, filed on Jun. 27, 2019.

(51) Int. Cl. G01N 33/72

(52) U.S. Cl. CPC G01N 33/721; G01N 2001/4038; G01N 2800/22; G01N 33/726

(57) ABSTRACT

A diagnostic system detects and/or measures hemoglobin and/or hemoglobin variants in blood of subject to determine hemoglobin levels, hemoglobin variants, and/or anemia in the subject.

### RELATED APPLICATION

This application claims priority from U.S. Provisional Application No. 62/867,318, filed Jun. 27, 2019, the subject matter of which is incorporated herein by reference in its entirety.

### GOVERNMENT FUNDING

This invention was made with government support under Grant Nos. R41DK119048, R44HL140739 awarded by The National Institutes of Health. The United States government has certain rights to the invention.

### TECHNICAL FIELD

The present invention is related to a diagnostic system, and particularly relates to a diagnostic system that includes an electrophoresis device that rapidly and easily perform blood analysis.

### BACKGROUND

Anemia, characterized by low blood hemoglobin (Hb) level, is among the world's most common and serious health conditions. Anemia has high prevalence affecting a third of the world's population or about 1.62 billion people with the heaviest morbidity and mortality among women and children living in low-and-middle-income countries. In sub-Saharan Africa, the prevalence of anemia among preschool children is extremely high, and has been reported to be as high as 91% in West Africa. Anemia can cause severe consequences including poor birth outcomes in women, decreased work productivity in adults, and impaired cognitive and behavioral development in children.

Genetic Hb disorders, such as sickle cell disease (SCD), are among the major causes for anemia globally. Inherited Hb disorders are carried by nearly 7% of the world's population, with the most structural Hb variants being the recessive β-globin gene mutations, β^{S} or S and β^{C} or C. SCD arises when these mutations are inherited homozygously (Hb SS or SCD-SS) or paired with another β-globin gene mutation, such as hemoglobin C (Hb SC or SCD-SC). SCD is associated with chronic hemolytic anemia causing the highest morbidity and mortality among genetic Hb disorders and is found predominantly in resource-constrained regions of sub-Saharan Africa and south-east Asia. More specifically, the structural Hb variants, such as hemoglobin S (Hb S), has reduced oxygen affinity compared with normal hemoglobin (Hb A), which exacerbates HbS polymerization and in return further reduces HbS oxygen affinity and causes anemia.

Anemia and SCD are inherently associated and are both are prevalent in the same regions of the world. Anemia and SCD-related complications can be mitigated by early diagnosis followed by timely intervention. Anemia treatment depends on the accurate characterization of the cause, such as inherited Hb disorders. Meanwhile, Hb disorders or SCD treatment requires close monitoring of blood Hb level and patient's anemia status. As a result, it is crucially important to perform integrated detection of blood Hb level, anemia status, and Hb variants, especially in sub-Saharan Africa, India, and south-east Asia where anemia and inherited Hb disorders are the most prevalent. Blood Hb level (in g/dL) is used as the primary indicator of anemia, while the presence of Hb variants in blood is the primary indicator of an inherited disorder. In resource-rich countries, standard clinical laboratory tests, namely complete blood count (CBC) and high-performance liquid chromatography (HPLC), are typically used in the diagnosis of anemia and inherited Hb disorders. However, advanced laboratory techniques require trained personnel and state-of-the-art facilities, which are lacking in countries where both anemia and Hb disorders are prevalent. Therefore, there is a need for affordable, portable, easy-to-use, accurate point-of-care tests that facilitate decentralized anemia detection and Hb variant identification.

### SUMMARY

Embodiments described herein relate to a diagnostic system and/or electrophoresis device as well as methods of using the system and device thereof for detecting and/or measuring hemoglobin levels and/or variant hemoglobin phenotypes (or hemoglobin variants) in blood of a subject, and particularly relates to a point-of-care diagnostic system for measuring hemoglobin levels and/or detecting hemoglobin variants, in a subject to determine blood hemoglobin concentration or levels and the presence hemoglobin variants in blood of the subject. In some embodiments, the diagnostic system can be used to measure hemoglobin levels and/or hemoglobin variants to determine whether a subject has or is suspected of having anemia.

In some embodiments, the diagnostic system for detecting hemoglobin levels and/or hemoglobin variants, includes a cartridge, an electrophoresis band detection module, and a processor.

In some embodiments, the cartridge can include an electrophoresis strip that is positioned within the cartridge and structured to receive hemolysate from a blood sample that is combined or mixed with a calibrator. The cartridge can include first and second electrodes configured to generate an electric field across the electrophoresis strip. The application of an electric field to the first and second electrodes induces migration and separation of bands of the calibrator and hemoglobin and/or separated hemoglobin phenotypes in the hemolysate delivered to the electrophoresis strip.

In some embodiments, an electrophoresis band detection module can be configured to optically detect and track the bands of the calibrator and hemoglobin and/or separated hemoglobin phenotypes in a region interest on the electrophoresis strip caused by the applied electric field and generate band detection data based on the detected and tracked bands of migrated and separated calibrator and hemoglobin and/or separated hemoglobin phenotypes.

In some embodiments, the processor receives and analyzes the band detection data to determine hemoglobin levels and/or detect hemoglobin variants and generate diagnostic results based on the detected hemoglobin level and/or hemoglobin variants.

In some embodiments, the processor compares the intensity of the band of hemoglobin to the intensity of the band of the calibrator in the region of interest to determine hemoglobin level in the sample.

In other embodiments, the processor can compare the position of band of the calibrator relative to the positions of the separated hemoglobin phenotypes in the region of interest to determine or detect the presence of hemoglobin variants.

In other embodiments, the processor can initially determine the level of hemoglobin and then subsequently hemoglobin variants. The processor can also determine whether the subject has or is at risk of anemia based on the determined level of hemoglobin and/or the detected hemoglobin variant.

In some embodiments, the electrophoretic mobility of the calibrator relative to the hemoglobin is such that calibrator band can achieve separation from the hemoglobin band in the region interest and prior to completion of separation of the hemoglobin phenotypes.

In some embodiments, the calibrator band can achieve separation from the hemoglobin band in less than about 2.5 minutes, less than about 2 minutes, or less than about 100 seconds upon application of the applied electric field to the electrophoresis strip.

In other embodiments, the bands of hemoglobin phenotypes are separated after the calibrator band achieves separation from the hemoglobin band and less than about 8 minutes upon application of the electric field.

In other embodiments, the calibrator can have a substantially different color in the visible spectrum than the hemoglobin on the electrophoresis strip to allow the band of the calibrator to be more readily distinguished from the hemoglobin band and/or hemoglobin phenotypes. For example, the calibrator comprises xylene cyanol.

In some embodiments, the hemolysate of the blood sample introduced into a sample loading port at an amount less than about 10 μL.

In other embodiments, a buffer solution can saturate the electrophoresis strip. The buffer solution can include tris/borate/EDTA buffer solution at a pH of 8.4.

Other embodiments described herein relate to a method of detecting anemia in a subject in need thereof. The method includes combining a blood sample obtained from a subject with a calibrator, which has a different electrophoretic mobility than hemoglobin in the blood sample. The combined blood sample and calibrator can then be introduced to an electrophoresis device that induces migration and separation of optically detectable bands of the calibrator and hemoglobin and/or separated hemoglobin phenotypes of the blood sample. The bands of the calibrator and hemoglobin and/or separated hemoglobin phenotypes can then be optically detected and tracked, and band detection data based on the detected and tracked bands of migrated and separated calibrator and hemoglobin and/or separated hemoglobin variant phenotypes can be generated. The hemoglobin level and/or hemoglobin variants can then be determined based on the determined hemoglobin level and/or hemoglobin variants. The determined hemoglobin level and/or hemoglobin variants is indicative of whether the subject has anemia.

In some embodiments, the intensity of the band of hemoglobin to the intensity of the band of the calibrator in a region of interest can be used to determine hemoglobin level.

In other embodiments, the processor can compare the position of band of the calibrator relative to the positions of the separated hemoglobin phenotypes in the region of interest to determine or detect the presence of hemoglobin variants.

The level of hemoglobin can also be determined before determining hemoglobin variant type.

In some embodiments, the calibrator can lyse the cells of the blood sample and have a substantially different color in the visible spectrum than the hemoglobin to allow the band of the calibrator to be more readily distinguished from the hemoglobin bands. For example, the calibrator can be xylene cyanol.

In some embodiments, the calibrator can have a substantially different electrophoretic mobility than the hemoglobin upon application of an applied electric field to an electrophoresis strip on which the combined blood sample and calibrator is placed. The electrophoretic mobility of the calibrator relative to the hemoglobin is such that calibrator band can achieve separation from the hemoglobin band prior to completion of separation of the hemoglobin variant phenotypes.

In some embodiments, the calibrator band can achieve separation from the hemoglobin band in less than about 2.5 minutes, less than about 2 minutes, or less than about 100 seconds upon application of the applied electric field.

In other embodiments, the hemoglobin variants are separated after the calibrator band achieves separation from the hemoglobin band and less than about 8 minutes upon application of the electric field.

### BRIEF DESCRIPTION OF THE FIGURES

FIGS. 1(A-B) illustrate (A) schematic view of diagnostic system in accordance with an embodiment described herein and (B) an exploded view of an example of a cartridge electrophoresis device.

FIG. 2 is a bottom view of the device of FIG. 1.

FIG. 3 is a front view of an indicating member of the device of FIG. 1.

FIG. 4 is a side view of the device of FIG. 1 and an enlarged portion thereof.

FIG. 5 is a section view of the device of FIG. 2 taken along line 5-5.

FIG. 6 is another side view of the device of FIG. 1 and an enlarged portion thereof.

FIG. 7 is a schematic illustration of the device of FIG. 1 in use.

FIG. 8 is flow chart showing a method in accordance with an embodiment.

FIG. 9 illustrates a schematic representation of the HbVA test process is shown. First, a drop of Blood (red) is mixed with Standard Calibrator (xylene cyanol, blue) and applied on the cellulose acetate paper in the cartridge (t=0). Within the first 2.5 minutes (t≤2.5 min), the total hemoglobin (red) and standard calibrator (blue) are electrophoretically separated, at which time blood Hb level (g/dL) and anemia status is determined by the algorithm. Next, hemoglobin variant separation occurs (t≤8 min), which is then analyzed to determine the presence of major hemoglobin variants and types in the blood sample (i.e., Hb A, F, S, and C). The entire electrophoresis process is tracked in real-time by computer vision and the captured data is analyzed by the deep learning artificial neural network (ANN) algorithm for integrated blood Hb level prediction, anemia detection, and Hb variant identification in a single test.

FIGS. 10(A-F) illustrate images and schematics showing an overview of HbVA integrated hemoglobin level prediction, anemia detection, and hemoglobin variant identification. (A) 2D representation of an HbVA test trajectory and computer vision in time (y-axis) and space (x axis), visually illustrating the full electrophoretic band separation process in a single image. Each pixel row of the image corresponds to a single one second frame, with time increasing from top (0 s) to bottom (480 s). For each point on the x axis we plot the total intensities for the two-color bands (red=Hb and blue=standard calibrator), summed across the y range of the region of interest (ROI). The ROI is illustrated in the inset for a representative video frame. (B) Computer vision process the band information and generate a time series vector ρ(t) for t=0 to 150, evaluating the relative intensity between Hb band and the standard calibrator band, as input for the trained ANN. (C) Hb variant is identified based on the final location Hb variant band at the end of the test (t=480 s). (D) Individual frames within the ROI at 3 representative time points during HbVA test. At 60 s, detectable separation initiated between Hb band and standard calibrator band due to their major mobility differences while the Hb band remain unseparated (Top frame, ρ₆₀=0.36). At 92 s, Hb band and standard calibrator band further separates thus increasing band separation resolution (Middle frame ρ₉₂=0.39). At 150 s, total hemoglobin starts to separate into hemoglobin variants due to their minor mobility differences (Bottom frame, ρ₁₅₀=0.30). (E) 3D intensity profile extracted from image acquired at t=92 s (solid horizontal line in A and middle image in C). (F) An example pattern of time series vector ρ(t) including pi to peso recognized by the trained ANN.

FIGS. 11(A-T) illustrate integrated Hb level prediction, anemia detection and Hb variant identification in 4 representative Hemoglobin Variant/Anemia (HbVA) tests on clinical samples at different Hb levels and Hb variant phenotypes. (A-D) The first row includes 2D representation of HbVA test band trajectories. (E-H) The second row illustrates a representative frame for each test from the image arrays used to generate relative intensity ratio time series vectors ρ(t), which are then utilized by the artificial neural network (ANN) to predict the Hb levels following the procedure outlined in FIG. 12. (I-L) The third row demonstrates the electropherogram corresponding to the image frames in the second row generated from the intensity profile envelopes. The Hb levels predicted by HbVA (red) are compared against the reference method complete blood count (CBC) reported results. (M-P) The fourth row demonstrate the frames utilized to identify Hb variants. (Q-T) The fifth row demonstrates the electropherogram generated according to the band information in the fourth row. Each column represents HbVA test result for each patient. First column: HbVA test result for patient at Hb level of 6.0 g/dL and with homozygous HbSS (sickle cell disease, SCD patient); Second column: HbVA test result for patient at Hb level of 10.3 g/dL and with heterozygous HbAS (SCD patient undergoing transfusion therapy); Third column: HbVA test result for patient at Hb level of 12.7 g/dL and with heterozygous Hb SC disease (hemoglobin C disease); Fourth column: HbVA test results for patient at Hb level of 14.5 g/dL and with homozygous HbAA (healthy subject). The HbVA Hb level prediction and anemia detection results are compared against the reference method complete blood count (CBC) reported results. The Hb variant identified by HbVA are compared against the reference method high performance liquid chromatography (HPLC) reported results. HbVA demonstrated agreement in Hb level prediction, anemia detection and Hb variant identification with reference standard methods CBC and HPLC. (Patient 1: HbVA: 5.8 g/dL, Anemia, Hb SS vs. CBC&HPLC: 6.0 g/dL, Anemia, Hb SS; Patient 2: HbVA: 9.6 g/dL, Anemia, Hb AS vs. CBC&HPLC: 10.3 g/dL, Anemia, Hb AS; Patient 3: HbVA: 12.8 g/dL, Anemia, Hb SC vs. CBC&HPLC: 12.7 g/dL, Anemia, Hb SC; Patient 4: HbVA: 13.8 g/dL, Non-anemia, Hb AA vs. CBC&HPLC: 14.5 g/dL, Non-anemia, Hb AA). These results demonstrate HbVA's capability of enabling integrated blood Hb level prediction and Hb variant identification.

FIGS. 12(A-B) illustrate plots showing robustness and reproducibility of Hemoglobin Variant/Anemia (HbVA) Hb level prediction: (A) Robustness test of HbVA Hb level prediction was tested with 10 repeated tests using the same sample, comparing variances between 2 users (demonstrated in the inset figures). No significant difference (p=0.29) was observed between Hb level predicted from user 1 HbVA tests (filled red round, Mean±Standard Deviation=12.3±0.4 g/dL, n=5, left inset) and Hb levels predicted from user 2 HbVA tests (opened red circle, Mean±Standard Deviation=12.0±0.6 g/dL, n=5, right inset). Hb level predicted by all 10 repeated tests demonstrated agreement of ±1.0 g/dL against the 12.7 g/dL Hb level reported by reference standard of complete blood count (CBC). (B) Reproducibility test of HbVA Hb level prediction was tested using 3 samples with low, middle and high Hb levels reported from CBC. Each sample was tested 3 times by both HbVA and CBC. The standard deviation of HbVA predicted Hb levels are within 4% CV across low, middle and high Hb levels (Low Hb level: HbVA: Mean±Standard Deviation=6.1±0.2 g/dL, CV %=3.8% Middle Hb level: Mean±Standard Deviation=10.5±0.1 g/dL, CV %=1.0% and High Hb level Mean±Standard Deviation=14.0±0.3 g/dL, CV %=2.1%). The HbVA predicted Hb levels also agree with the reference standard CBC reported Hb levels (Low Hb level: Mean±Standard Deviation=6.0±0.3 g/dL, CV %=5.0%; Middle Hb level: Mean±Standard Deviation=10.4±0.1 g/dL, CV %=1.0%; High Hb level: Mean±Standard Deviation=14.6±0.3 g/dL, CV %=2.1%). HbVA predicted Hb levels from all 3 groups of tests were within ±0.6 g/dL and ±5.0% with the CBC reported. No significant difference was found between the Hb level measured using HBVA against reference method CBC through tested Hb range (p=0.76, 0.29 and 0.08, respectively). n=3 for each test.

FIGS. 13(A-C) illustrate plots showing an Hemoglobin Variant/Anemia (HbVA) artificial neural network (ANN) based deep learning algorithm accurately predicts Hb levels. (A) HbVA measures blood Hb levels are strongly associated with CBC measured results (PCC=0.95, p<0.001). The dashed line represents the ideal result where HbVA Hb level is equal to the CBC Hb level whereas solid line represents the actual data fit. (B) Bland-Altman analysis reveals HbVA predicts blood Hb levels to within ±0.55 of the Hb level (absolute mean error) with minimal experimental bias with −0.1 g/dL, indicating that Hb prediction has very small bias. The dashed light grey line indicates the relationship between the residual and the average Hb level measurements obtained from the CBC and HBVA (r=−0.07). The dashed dark grey line represents 95% limits of agreement (±1.5 g/dL). (C) The receiver-operating characteristic (ROC) analysis graphically illustrates HbVA's performance against a random chance diagnosis (grey line), with an area under the curve of 0.99, and a perfect diagnostic (green lines), with an area under the curve of 1. The area under the curve of 0.99 suggests HbVA's viable diagnostic performance. n=46.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “microchannels” as used herein refer to pathways through a medium (e.g., silicon) that allow for movement of liquids and gasses. Microchannels thus can connect other components, i.e., keep components “in liquid communication.” While it is not intended that the present invention be limited by precise dimensions of the channels, illustrative ranges for channels are as follows: the channels can be between 0.35 and 100 μm in depth (preferably 50 μm) and between 50 and 1000 μm in width (preferably 400 μm). Channel length can be between 4 mm and 100 mm, or about 27 mm. An “electrophoresis channel” is a channel substantially filled with a material (e.g., cellulose acetate paper) that aids in the differential migration of biological substances (e.g., for example whole cells, proteins, lipids, nucleic acids). In particular, an electrophoresis channel may aid in the differential migration of blood cells based upon mutations in their respective hemoglobin content.

The term “microfabricated”, “micromachined” and/or “micromanufactured” as used herein, means to build, construct, assemble or create a device on a small scale (e.g., where components have micron size dimensions) or microscale. In one embodiment, electrophoresis devices are microfabricated (“microfabricated electrophoresis device”) in about the millimeter to centimeter size range.

The term “polymer” refers to a substance formed from two or more molecules of the same substance. Examples of a polymer are gels, crosslinked gels and polyacrylamide gels. Polymers may also be linear polymers. In a linear polymer the molecules align predominately in chains parallel or nearly parallel to each other. In a non-linear polymer the parallel alignment of molecules is not required.

The term “electrode” as used herein, refers to an electric conductor through which an electric current enters or leaves.

The term “channel spacer” as used herein, refers to a solid substrate capable of supporting lithographic etching. A channel spacer may comprise one, or more, microchannels and is sealed from the outside environment using dual adhesive films between a top cap and a bottom cap, respectively.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk of” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a compound or sample. In one respect, a compound or sample may be derived from an organism or particular species.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

Embodiments described herein relate to a diagnostic system and electrophoresis device as well as methods of using the system and device thereof for detecting and/or measuring hemoglobin levels and/or variant hemoglobin phenotypes (or hemoglobin variants) in blood of subject, and particularly relates to a point-of-care diagnostic system for measuring hemoglobin levels and/or detecting hemoglobin variants, in a subject to determine blood hemoglobin concentration or levels and the presence hemoglobin variants in blood of the subject. In some embodiments, the diagnostic system can be used to measure hemoglobin levels and hemoglobin variants to determine whether a subject has or is suspected of having anemia.

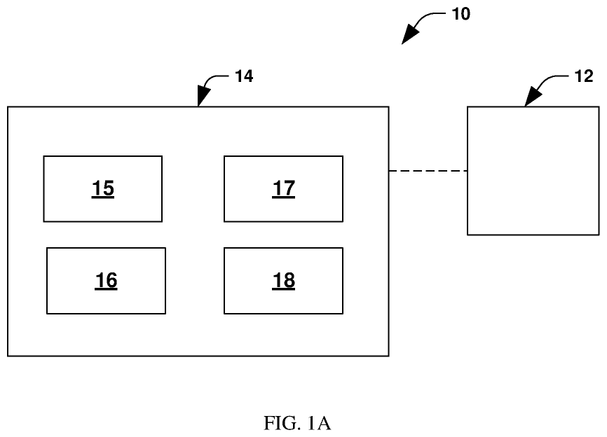

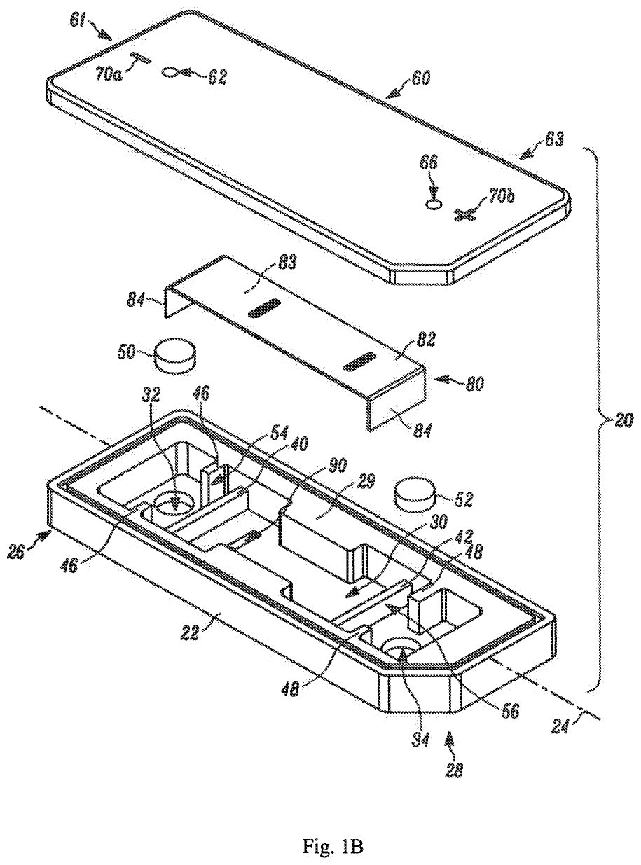

FIG. 1A is a schematic illustration of a point-of-care blood diagnostic system 10 in accordance an embodiment described herein. A point-of-care diagnostic system includes devices that are physically located at the site at which patients are tested and sometimes treated to provide quick results and highly effective treatment. Point-of-care devices can provide information and help in diagnosing patient disorders while the patient is present with potentially immediate referral and/or treatment. Unlike gold standard laboratory-based blood testing for disorders, the disclosed point-of-care devices enable diagnosis close to the patient while maintaining high sensitivity and accuracy aiding efficient and effective early treatment of the disorder and/or infection.

The diagnostic system 10 includes a cartridge electrophoresis device 12 for performing electrophoresis analysis on a sample, and a reader 14 that can interface with the cartridge 12 to perform electrophoresis, detect and track electrophoresis bands of hemoglobin and a calibrator applied to the electrophoresis device, receive and analyze detected and tracked bands, and optionally convey and/or display the result to a user of the system 10.

Referring to FIGS. 1B-3, an example cartridge electrophoresis device 20 includes a housing 22, an indicating member 80, and a cover 60. The housing 22 has a generally rectangular shape and extends along a centerline 24 from a first end 26 to a second end 28. A wall 29 of the housing 22 defines a recessed channel 30, i.e., microchannel, which extends between the first and second ends 26, 28. The microchannel 30 can be between about 0.35 and 100 μm in depth (preferably 50 μm) and between about 50 and 1000 μm in width (preferably 400 μm). The microchannel 30 length along the centerline 24 can be between 4 mm and 100 mm (preferably 27 mm).

The microchannel 30 is constructed to receive an electrophoresis strip sieving medium that aids in the differential migration of biological substances, such as whole cells, proteins, lipids, and nucleic acids. More specifically, the electrophoresis strip in the microchannel 30 is configured to suppress convective mixing of the fluid phase through which electrophoresis takes place and contributes to molecular sieving. In one example, the electrophoresis strip can constitute cellulose acetate paper. The electrophoresis channel 30 can aid in the differential migration of hemoglobin variants or types from a hemolysate of blood of a subject.

The wall 29 also helps to define first and second buffer pools 32, 34 located at opposite ends of the channel 30. More specifically, the first buffer pool 32 is positioned at or adjacent to the first end 26 of the housing 22. The second buffer pool 34 is positioned at or adjacent to the second end 28 of the housing.

A first opening 36 extends through the bottom of the housing 22 into the first buffer pool 32. A second opening 38 extends through the bottom of the housing 22 into the second buffer pool 34. As shown, the openings 36, 38 are circular. Alternatively, the openings 36, 38 can have any round or polygonal shape. In any case, an electrode 50 is positioned in the first opening 36 and exposed to the first buffer pool 32. An electrode 52 is positioned in the second opening 38 and exposed to the second buffer pool 34. The electrodes 50, 52 can be made from a conductive material, e.g., steel, 300 stainless steel, graphite and/or carbon.

A wall 40 is positioned within the microchannel 30 and helps define the boundary of the first buffer pool 32. The wall 40 spans the entire width of the microchannel 30 perpendicular to the centerline 24. A pair of restricting members 46 extends from the wall 29 of the housing 22 towards the centerline 24. The restricting members 46 extend parallel to the wall 40 and are positioned closer to the first end 26 than the wall. The restricting members 46 are spaced from one another and spaced from the centerline 24. A gap or space 54 is formed between the restricting members 46 and the wall 40.

A wall 42 is positioned within the microchannel 30 and helps define the boundary of the second buffer pool 34. The wall 42 spans the entire width of the microchannel 30 perpendicular to the centerline 24. A pair of restricting members 48 extends from the wall 29 of the housing 22 towards the centerline 24. The restricting members 48 extend parallel to the wall 42 and are positioned closer to the second end 28 than the wall 42. The restricting members 48 are spaced from one another and spaced from the centerline 24. A gap or space 56 is formed between the restricting members 48 and the wall 42.

An opening 90 extends through the bottom (as shown) of the housing 22 into the microchannel 30 and between the walls 40, 42. The opening 90 can have any shape but regardless is used as a sample loading port by which blood samples can be injected or otherwise supplied to the microchannel 30, as will be described.

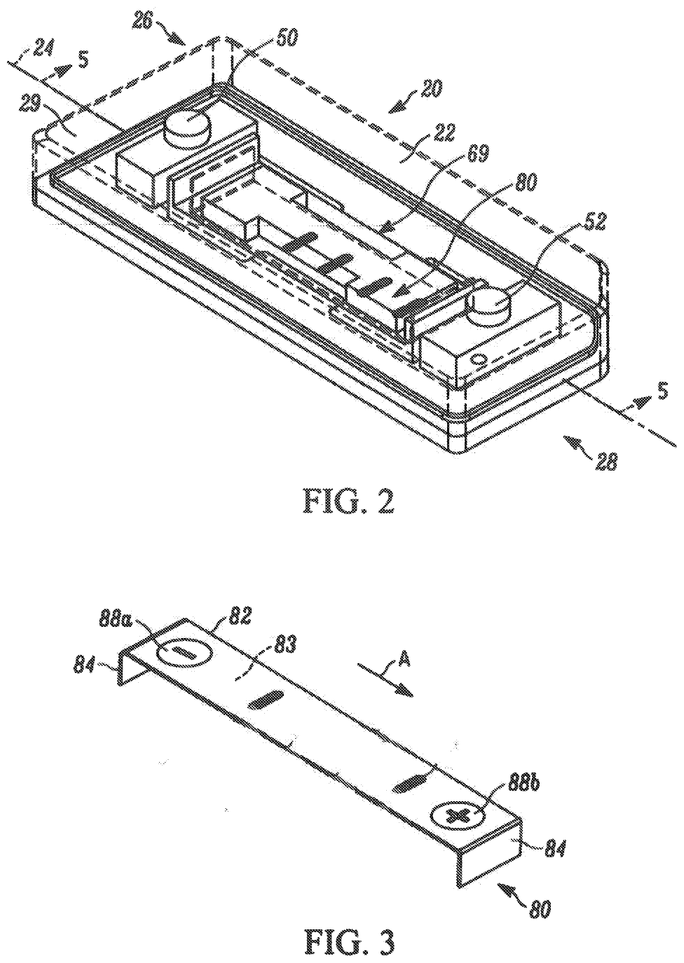

The indicating member 80 is elongated and includes a base 82 and a pair of legs 84 extending from the base. The indicating member 80 can be formed from the electrophoresis strip. Optionally, the electrophoresis strip 80 can be secured to or embedded in a hard, conductive material, e.g., metal, as indicated generally in phantom at 83 in FIGS. 1-2. In any case, the indicating member 80 is generally rectangular with the legs 84 extending at an angle, e.g., perpendicular, from the base 82. Consequently, the indicating member 80 can have a U-shaped construction.

A pair of electrode indications 88a, 88b is provided at opposite ends of the base 82. As shown, the indication 88a is a negative (−) terminal indication and the indication 88b is a positive (+) terminal indication. The terminal designations could, however, be reversed.

Optionally, a series of test indications (not shown) can be provided along the base 82 between the electrode indications 88a, 88b and parallel to the length of the microchannel 30. Depending on the blood test to be performed, the test indications can be symmetrically or asymmetrically spaced from one another along the base 82. The test indications can be longitudinally aligned with one another or misaligned. The test indications can have the same dimensions or different dimensions from one another. In one example, the test indications are colored bands indicative of the basic types of hemoglobin, e.g., normal hemoglobin (HbA), fetal hemoglobin (HbF), sickle hemoglobin (HbS), hemoglobin C/E/A (HbC or HbA₂).

The cover 60 is shaped similarly to the housing 22 and extends from a first end 61 to a second end 63. The cover 60 is generally rectangular and configured to be secured to the housing 22. The cover 60 can, for example, form a snap-fit connection with the housing 22. In any case, the cover 60 cooperates with housing 22 to define and enclose the microchannel 30.

A first opening 62 extends through the first end 61 and a second opening 66 extends through the second end 63. A first recess 64 is formed in the underside of the cover 60 and extends to the opening 64. A second recess 68 is formed in the underside of the cover 60 and extends to the opening 66.

The underside of the cover 60 further includes a plurality of support members 74 positioned between the openings 62, 66. As shown, the support members 74 are rectangular projections extending parallel to one another and perpendicular to the length of the cover 22.

A portion 69 of the housing 22 between the ends 26, 28 is transparent or optically clear to define an optical window that allows light to pass into and/or through a portion of the microchannel 30, e.g., the test indications 86 on the indicating member 80 when positioned within the microchannel. The ability to pass light can be a necessary step during analysis of a patient sample within the cartridge 20. The optical window 69 can be a material and/or construction that necessarily or desirably alters light entering the optical window 69 as a part of the analysis of the patient sample within, such as collimating, filtering, and/or polarizing the light that passes through the optical window 69. Alternatively, the optical window 69 can be transparent or translucent, or can be an opening within the housing 22 of the cartridge 20. The cartridge 20 can include a reflector (not shown) opposite the optical window 69 that reflects the incoming light back through the optical window 69 or through another optical window, or can include a further optical window opposite the light entry window to allow light to pass through the cartridge 20.

To this end, the portion or optical window 69 can optionally be provided with a hydrophilic coating to prevent spotting or hazing on the portion 69. The cover 60 and the housing 22 can both be formed from hard, durable materials, such as a plastic, polymer and/or glass.

When the device is assembled, the electrodes 50, 52 are positioned in the openings 36, 38 and extend into each buffer pool 32, 34. The legs 84 of the indicating member 80 are inserted into the gaps 54, 56 at each end 26, 28 of the housing 22 such that the indicating member 80 extends parallel to/along the housing centerline 24 within or adjacent to the microchannel 30. The restricting members 46, 48 and walls 40, 42 are longitudinally spaced from one another in a manner that prevents or limits longitudinal movement of the indicating member 80 relative to the housing 22. More specifically, the indicating member 80—in particular the electrophoresis strip, e.g., cellulose acetate paper—has a length substantially equal to the longitudinal distance between the restricting members 46, 48 such that the indicating member abuts the restricting members to prevent relative longitudinal movement therebetween.

The first buffer pool 32 and the second buffer pool 34 each receive a buffer solution 51, 53 that at least partially saturates the indicating member 80 extending into the respective pool. The buffer solution 51, 53 can exhibit an affinity to hemoglobin and a calibrator mixed with a blood sample that is applied to the electrophoresis strip. The pH induced net negative charges of the hemoglobin variant phenotypes and calibrator cause them to travel from the negative electrode to the positive electrode when an electric field is applied to the electrophoresis strip.

In some embodiments, the buffer solution can provide ions for electrical conductivity at mild basic pH, for example, a pH of about 7.5 to about 8.7, (e.g., pH 8.4). Optionally, a generally used additive may be added to the above-mentioned buffer solution. Examples thereof include surfactants, various polymers, hydrophilic low-molecular-weight compounds, and the like. By way of example, the buffer solution can include Tris/Borate/EDTA solution, at a pH of 8.4. The buffer pools 32, 34 can each receive about 1 μL to about 200 μL of the respective buffer solution 51, 53.

The cover 60 is secured to the housing 22 to confine the indicator member 80 within the microchannel 30. The opening 62 in the cover 60 is aligned with the electrode 50 within the first buffer pool 32. The negative electrode indication 88a is generally positioned between the opening 62 and the electrode 50. The opening 66 in the cover 60 is aligned with the electrode 52 within the second buffer pool 34. The negative electrode indication 88b is generally positioned between the opening 66 and the electrode 52.

When the cover 60 is secured to the housing 22 the optical window 69 of the housing is aligned with the indicating member 80 such that all the test indications 86 on the electrophoresis strip are visible through the optical window 69. In other words, the support members 74 on the cover 60 do not visually obstruct the test indications 86 through the optical window 69.

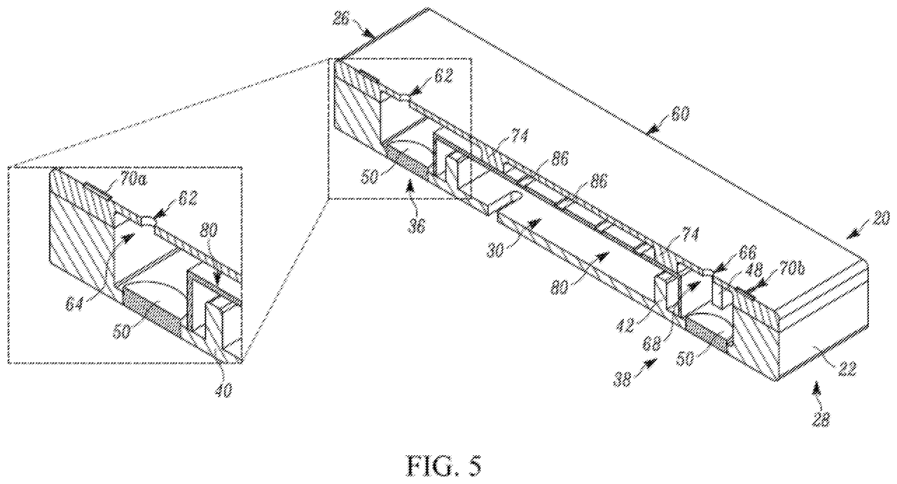

Referring to FIG. 5, an electrode 94 is inserted through the opening 62 in the cover, into the first buffer pool 32, and into contact with the electrode 50. An electrode 96 is inserted through the opening 66 in the cover, into the second buffer pool 34, and into contact with the electrode 52. The electrodes 94, 96 are electrically connected to a power supply 98. The electrodes 50, 52, 94, 96, buffer solutions 51, 53 and indicating member 80 cooperate to form an electrical circuit through the cartridge electrophoresis device 20.

The power supply 98 is capable of generating an electric field of about 1V to about 400V. In some instances, the voltage applied to the cartridge electrophoresis device 20 by the electrodes 94, 96 does not exceed 250V. Regardless, an electric field is generated across the electrophoresis strip of the indicating member 80 effective to promote migration of hemoglobin variants in a blood sample along the electrophoresis strip.

A patient sample, such as patient blood sample can be combined with a calibrator and provided in the cartridge 20 and on the electrophoresis strip using a sample applicator 92. The sample applicator 92 provides a more precise and/or controlled deposition of the sample onto the electrophoresis strip. The calibrator mixed with the blood sample can assist with the electrophoresis process and/or assist with interpreting the electrophoresis results.

For example, the blood sample can be mixed with one or more calibrators or electrophoretic markers that have known migrations rates and/or distances for a given applied voltage and/or voltage application time. These calibrators can normalize the results of the electrophoresis process by having different electrophoretic mobilities or migration rates relative to hemoglobin in the blood sample, thereby reducing the effects of sample-to-sample variability. These calibrators can assist with evaluating the resultant banding of the patient sample.

The calibrator can be either positively or negatively charged so it can migrate along the electrophoresis strip upon application of the electric field and form a band that can be optically detected by the band detection module 14 of the system 10. In some embodiments, the calibrator can have a negative charge at pH of the buffer solution.

In some embodiments, the calibrator can have a substantially different electrophoretic mobility than the hemoglobin upon application of the electric field to the electrophoresis strip. The electrophoretic mobility of the calibrator relative to the hemoglobin is such that calibrator band can achieve separation from the hemoglobin band in the region interest and prior to completion of separation of the hemoglobin phenotypes.

In some embodiments, the calibrator band can achieve separation from the hemoglobin band in less than about 2.5 minutes, less than about 2 minutes, less than about 100 seconds, or from about 60 seconds to about 2.5 minutes, upon application of the electric field to the electrophoresis strip and before the hemoglobin band starts to separate based on phenotype.

In other embodiments, the bands of hemoglobin phenotypes are separated after the calibrator band achieves separation from the hemoglobin band and less than about 8 minutes upon application of the electric field.

In other embodiments, the calibrator can have a substantially different color in the visible spectrum and/or other electromagnetic radiation frequencies than the hemoglobin on the electrophoresis strip to allow the band of the calibrator to be more readily distinguished from the hemoglobin band and/or hemoglobin phenotype bands. Calibrators having different colors than hemoglobin (Red) can include for example, bromocresol green, bromocresol purple, bromophenol blue, bromothymol blue, m-Cresol purple, orange g, and xylene cyanol.

In some embodiments, the calibrator can have a substantially different color in the visible spectrum than the hemoglobin and upon combination or mixing with the blood sample can lyse the cells of the blood sample. An example of such a calibrator is xylene cyanol.

It will be appreciated that other electrophoretic markers that have a similar or different electrophoretic mobility than the hemoglobin can be added to the blood sample. The other markers can have the same or a different color than hemoglobin and have molecular weights that vary between 200 g/mol to 1,000 g/mol, for example, about 300 g/mol to about 800 g/mol, or about 400 g/mol to about 700 g/mol.

A sample applicator 92 is filled with hemolysate of a blood sample BS and calibrator and inserted into the loading port 90 on the underside of the housing 22. The hemolysate of the blood sample BS and calibrator introduced into the loading port 90 can be, for example, less than about 10 μL. The cartridge electrophoresis device 20 can therefore be microengineered and capable of processing a small volume, e.g., a finger or heel prick volume.

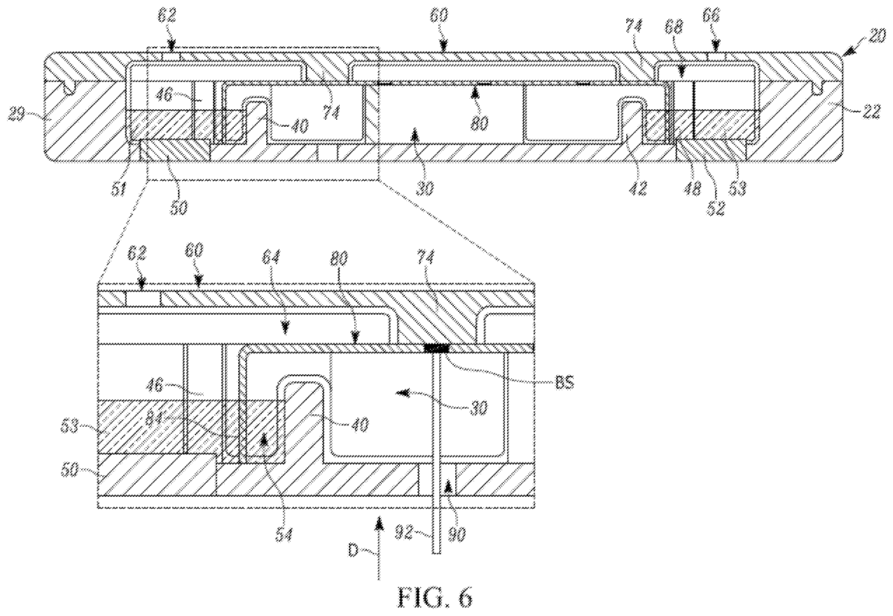

In any case, the sample applicator 92 is urged in the direction D towards the indicating member 80. The loading port 90 therefore helps to guide the sample applicator 92 towards a desired location on the indicating member 80. Since the indicating member 80 is formed from the electrophoresis strip, the sample applicator 92 can be inserted to the strip and release the sample therein to the left [as shown in FIG. 6] of all the test indications 86.

The loading port 90 is aligned with and extends towards one of the support structures 74. As a result, the support members 74—especially the leftmost support member—acts as a reaction surface to the indicating member 80 as the sample applicator 92 extends into the electrophoresis strip and deposits the sample therein. The support member 74 thereby prevents movement of the indicating member 80 away from the moving sample applicator 92. This helps prevent deformation or distortion of the indicating member 80 and helps the user release the sample in the proper location along the indicating member.

Once the sample is released in position, the sample applicator 92 is withdrawn (in a direction opposite D) from the electrophoresis device 20. Capillary action by the electrophoresis strip 80 can maintain the sample in position. The power supply 98 is actuated/turned on, which supplies current to the buffer pools 32, 34 and indicating member 80 as described, which establishes a continuous electrical path through the device 20 via the buffer pools 32, 34 and cellulose acetate paper 80 in contact therewith.

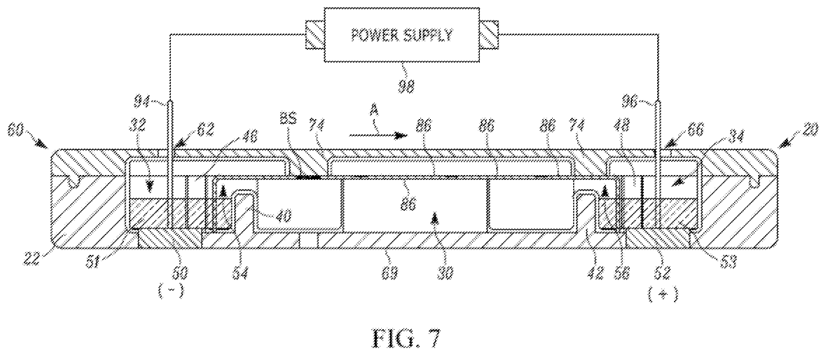

In this example, forming the indicating member 80 out of cellulose acetate paper allows the indicating member to also act as the sieving medium during the electrophoresis process. To this end, hemoglobin in the hemolysate of the blood sample and the calibrator migrate through the indicating member 80 and towards the positive electrode 52 in the direction indicated by A (FIG. 7).

With the patient sample in place, a voltage is applied using the electrodes, causing hemoglobin and the calibrator to migrate across the electrophoresis strip over a defined time. The hemoglobin and calibrator will separate initially into a single hemoglobin band representing or indicative of the level or amount of hemoglobin in the sample and a calibrator band, and then the single hemoglobin band will separate into hemoglobin phenotype bands due to the applied voltage and the physical and electrical properties of the various hemoglobin phenotypes. One or all of the applied voltage, current and the application time can be predetermined or preset based on the various parameters of the electrophoresis testing being performed. Alternatively, one or more of the voltage, current and application times can be variable and based on the banding of the hemoglobin and calibrator therein. For example, the movement of a calibrator added to the patient sample can be monitored as the calibrator moves across the electrophoresis strip. That is, imaging/monitoring of the electrophoresis testing, and/or the calibrators thereon, can be performed in a continuous or timed interval manner during the testing process. For example, images of the electrophoresis process can be continuously captured, such as by a video imaging process, or the images can be captured at regular intervals based on time and/or the distance one or more bands have traveled. Once the calibrator has reached a predetermined location across the electrophoresis strip, the test can be terminated with the removal of the applied voltage.

The bands of the calibrator and hemoglobin and/or separated hemoglobin phenotypes and variants (A, F, S, and C) and can then be optically detected and tracked and band detection data based on the detected and tracked bands of migrated and separated calibrator and hemoglobin and/or separated variant hemoglobin phenotypes can be generated. Hemoglobin level and/or variant hemoglobin phenotypes can then be determined based on the detected hemoglobin level and hemoglobin variant phenotypes. The determined hemoglobin level and/or detected hemoglobin variants is indicative of whether the subject has anemia.

Depending on the distribution of the hemoglobin and/or hemoglobin phenotypes relative to relative to the calibrator a diagnosis regarding the sample can be made. To this end, the transparent portion 69 of the housing 22 allows the reader to simply visualize the distribution of hemoglobin relative to the calibrator on the indicating member 80.

The configuration of the cartridge electrophoresis device 20 is advantageous for several reasons. First, as noted, the support structures 74 provided on the cover 60 help prevent movement of the indicating member 80 while the sample BS is injected/provided into the cellulose acetate paper forming the indicating member. Second, the walls 40, 42 and associated restricting members 46, 48 each help prevent or limit relative movement between the indicating member 80 and the housing 22 during loading and operation of the device 20.

Moreover, the wall 40 also helps to prevent the buffer solution 51 within the first buffer pool 32 from leaking into the microchannel 30 via capillary action. Similarly, the wall 42 helps to prevent the buffer solution 53 within the second buffer pool 34 from leaking into the microchannel 30 via capillary action. The walls 40, 42 help ensure current flow through the device 20 is continuous and helps the device maintain a substantially constant pH during operation.

Additionally, embedding the electrodes 50, 52 within the bottom of the buffer pools 32, 34 helps to ensure a consistent supply of electric field through the cellulose acetate paper on the indicating member 80, even when/if either buffer solution 51, 53 begins to evaporate.

The reader 14 can include a housing (not shown) that surrounds and encloses some portion or all of the reader components. The housing of the reader 14 is constructed of materials, which may involve a suitably robust construction such that the reader 14 is rugged and portable. Alternatively, the reader 14 can be designed and/or constructed for use in a permanent or semi-permanent location, such as in a clinic or laboratory.

The housing of the reader 14 includes a cartridge interface that interacts with and/or engages the cartridge 12 for analysis of a patient sample. The cartridge interface can be a slot that is shaped to receive the cartridge 12. Alternative designs and/or structures of cartridge interfaces can be used with the reader 14.

Referring to FIG. 1, the reader 14 can include an electrophoresis module 15 that can interface with the cartridge 12 to perform the electrophoresis test. The electrophoresis module 15, alone or in conjunction with processing circuitry, can control the electrophoresis test, including voltage/current application time and/or level. The electrophoresis module can supply electrical power from the power supply 98 to the cartridge 12, or electrophoresis strip, directly, to establish the necessary voltage across the electrophoresis strip for testing. The voltage can be applied at a higher level to increase the speed of the testing, however, the increased speed can cause decreased band fidelity, which can increase the difficulty and error of the band analysis and evaluation. A lower applied voltage can increase band fidelity but can lengthen the required testing time. Alternatively, the electrophoresis module 15 can vary the applied voltage or current, while maintaining the other stable, to achieve a desired or required level of band fidelity and testing speed. For example, an initial test to identify a patient condition can be carried out at a higher level voltage level to speed the test and a subsequent test to quantify the condition can be carried out a lower voltage level to generate clearer or more accurate results.

An electrophoresis band detection module 16, alone or in conjunction with the electrophoresis module 15, can capture, analyze and/or evaluate the electrophoresis test results and/or any other band detection characteristic(s) related to or otherwise based on the electrophoresis test results. The electrophoresis band detection module 16 can include an imaging device, such as a digital image sensor, to capture or optically detect the electrophoresis strip and the banding thereon. The band detection module can optically detect and track the bands of the calibrator and hemoglobin and/or separated hemoglobin phenotypes in a region interest on the electrophoresis strip caused by the applied electric field and generate band detection data based on the detected and tracked bands of migrated and separated calibrator and hemoglobin and/or separated hemoglobin phenotypes.

The reader 14 can also include an output 17 that includes one or more visual and/or audible outputs although in other examples the output is data and does not include visual and/or audible outputs. The output 17 communicates information regarding the status of the reader 14, the results of analysis of a patient sample, instructions regarding use of the reader 14 and/or other information to a user or other computing device. The output 17 can include a display, such as a screen, such as a touchscreen, lights, and/or other visual indicators. The touchscreen used to display information, such as analysis results, to the user can also be used by a user to input to the reader 14. Alternative interfaces can be included on and/or connected to the reader 14, such as a keyboard and/or mouse. Additionally, user devices, such as a cellphone or tablet, can be connected to the reader 14 to provide an interface portal through which a user can interact with the reader 14. The output 17 can include a speaker, buzzer, or other audible indicators. The output 17 can be output through an external device, such as a computer, speaker, or mobile device connected physically and/or wirelessly to the reader 14. The output 17 can output data, including the collected analysis data and/or interpretative data indicative of the amount of hemoglobin and various hemoglobin phenotypes within the patient blood sample. The interpretive data output can be based on the analysis data collected and processed by the processing circuitry of the reader 14.

The reader can further include a processor or processing module 18. The processor 18 can receive inputs or band detection data from the electrophoresis band detection module 16. In some embodiments, the processor receives and analyzes the band detection data to determine hemoglobin level and/or hemoglobin variants present in the blood sample and generate diagnostic results based on the hemoglobin level and hemoglobin variant phenotypes.

In some embodiments, the processor compares the intensity of the band of hemoglobin to the intensity of the band of the calibrator in the region of interest to determine hemoglobin level in the sample.

In other embodiments, the processor can compare the position of band of the calibrator relative to the positions of the separated hemoglobin phenotypes in the region of interest to determine or detect the presence of hemoglobin variants.

In other embodiments, the processor can initially determine the level of hemoglobin and then subsequently detect hemoglobin variant phenotype. The processor can also determine whether the subject has or is at risk of anemia based on the determined level of hemoglobin and the detected hemoglobin variants.

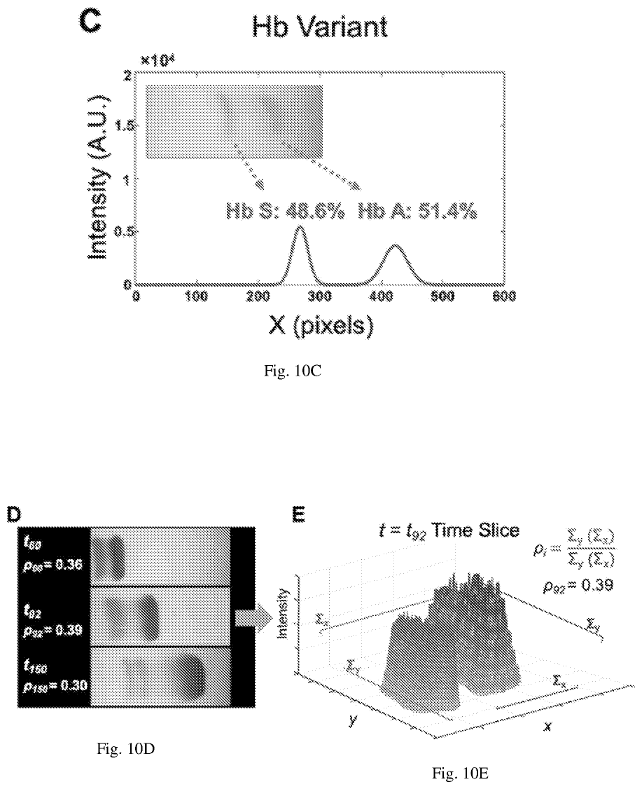

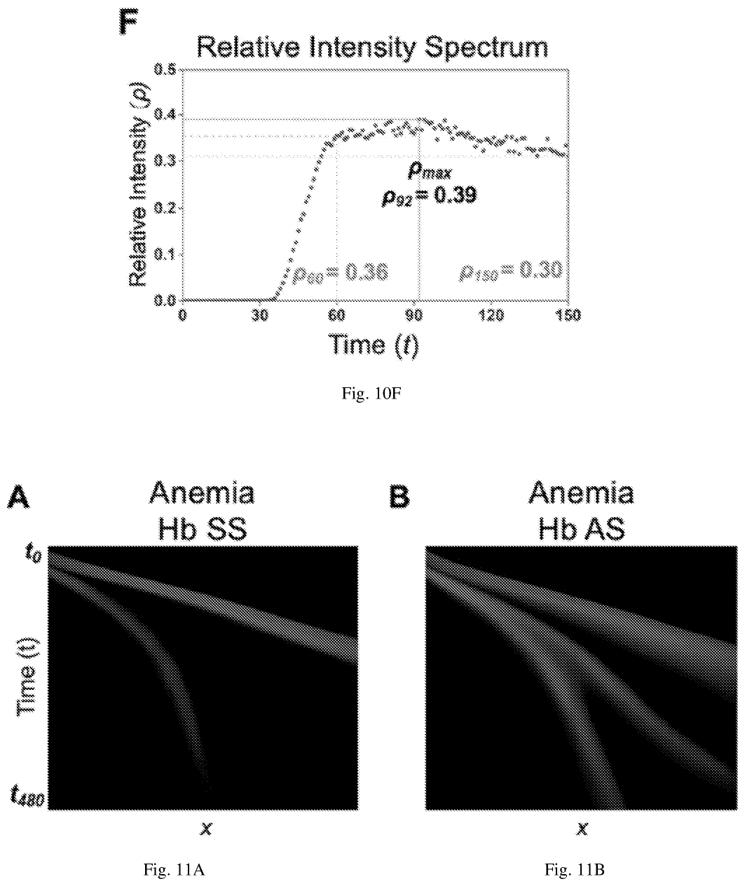

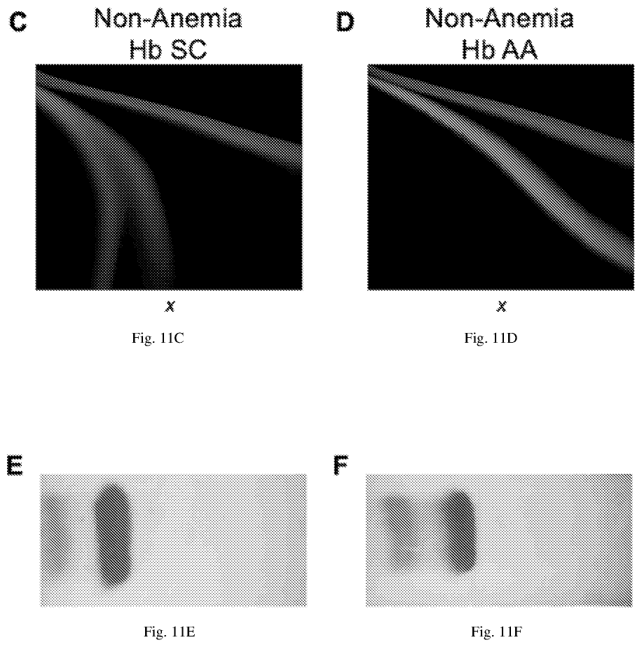

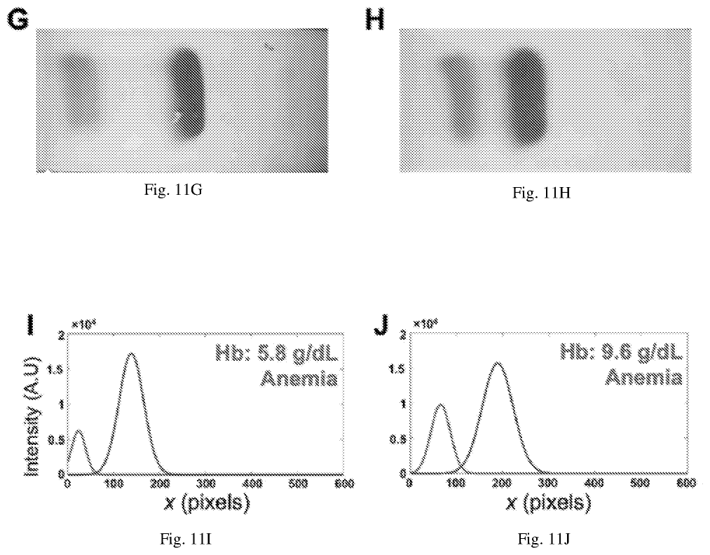

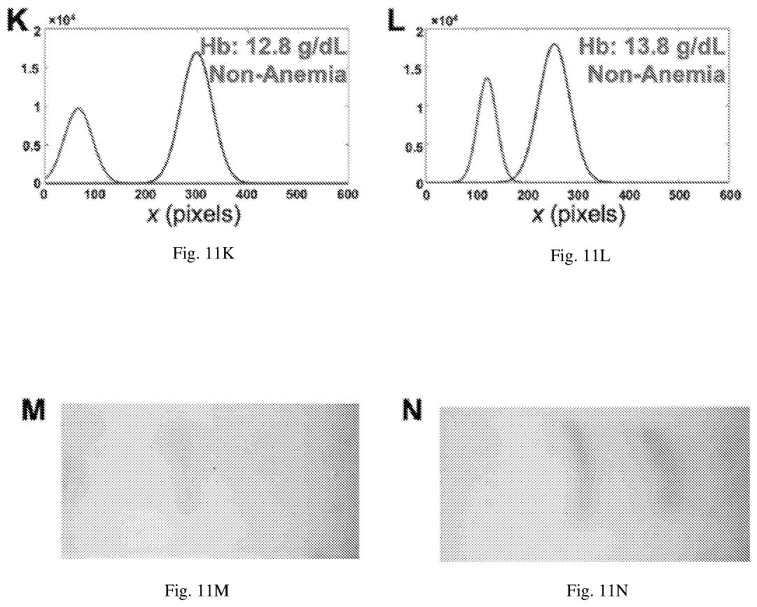

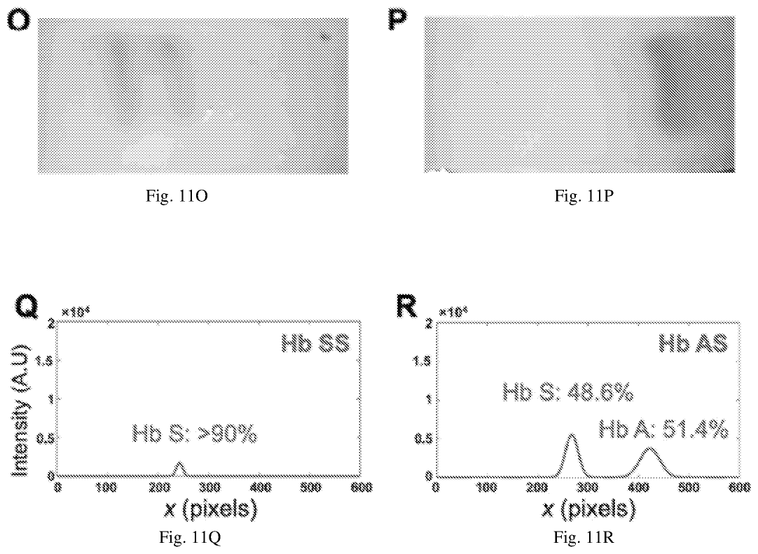

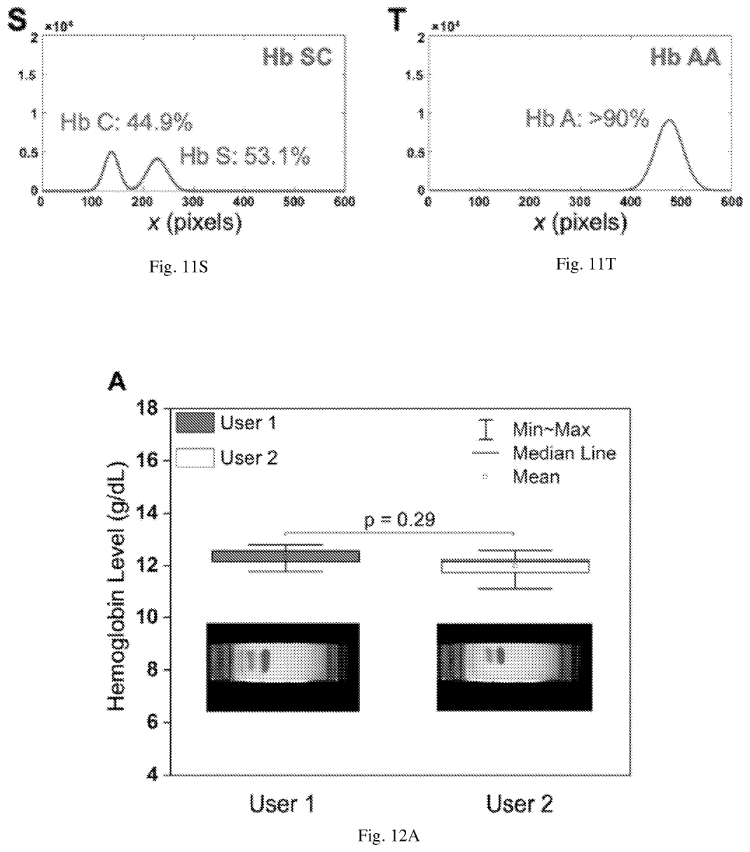

By way of example, the electrophoresis band detection module can automatically identify hemoglobin and calibrator band position and define a region of interest (ROI) (FIG. 10A) specific for each test and track the band movement within the ROI (FIG. 10A). The processor then processes the band information and generates a time series vector ρ(t), evaluating the relative intensity between hemoglobin band and the calibrator band, as input for a trained artificial neural network (ANN) of the processor (FIG. 10B). The trained ANN performs pattern recognition and regression analysis by examining underlying input data using the time series vector ρ(t) and predicts the hemoglobin level in g/dL (FIG. 10B) as well as detects anemia over broad hemoglobin level range (FIG. 10A-L). This is followed by the second step of hemoglobin variant identification. Using late time (t₁₅₀→t₄₈₀) results from the same test, hemoglobin variants are can be identified by a hemoglobin variant algorithm (FIG. 10C, FIG. 11M-T). More specifically, the time series vector ρ(t) encoding the relative intensity, ρᵢ, obtained using the information within ROI during the first 150 s in the electrophoresis test (FIG. 10D), can be evaluated as the relative intensity ratio of the total hemoglobin band intensity over total standard calibrator band intensity for each frame (FIG. 10E&F). The trained ANN analyzes and recognizes the underlying pattern of the relative hemoglobin intensity pi and associates this pattern with a hemoglobin level and corresponding anemia status. Within the same test, the algorithm then tracks Hb variant band migration and identifies hemoglobin variants based on their final locations at the end of test (FIG. 2C, t≤8 min). In case of a single hemoglobin variant detected, such as HbSS (FIG. 11M&Q) or HbAA (FIG. 11P&T), the algorithm reports a percentage value greater of >90%. If there is more than one peak identified, then the areas under each of the peaks are calculated and the relative percentages are reported, for example in the cases of HbAS (FIG. 11N&R), HbSC (FIG. 11O&S).

The output from the processor 18 can be transmitted through the output 17 of the reader 14 or transmitted to an external device and/or system, such as a computer, mobile device, and remote server or database.

In other embodiments, the electrophoresis band detection module can include a mobile phone imaging system to visualize and quantify hemoglobin variant migration. For example the mobile phone imaging system can include a mobile telephone that is used to image hemoglobin variant migration and a software application that recognizes and quantifies the hemoglobin band variant types to make a diagnostic decision. The hemoglobin band types can include hemoglobin types C/A, S, F, A2.

In some embodiments, the diagnostic system can be used to diagnose whether the subject has hemoglobin variants HbAA, HbSS, HbAS, and HbSC. In other embodiments, the diagnostic system can be used to diagnose whether the subject has or an increased risk of anemia.

In some embodiments, the diagnostic system can be used in a method where hemolysate of a blood sample from a subject is combined with a calibrator as described herein and introduced into a sample loading port. The blood sample includes hemoglobin. Hemoglobin bands formed on the cellulose acetate paper are then imaged with the imaging system to determine hemoglobin phenotype for the subject. The hemoglobin phenotype can be selected from the group consisting of HbAA, HbAA, HbSS, HbSC, and HbA2.

One advantage of the diagnostic system described herein, and particularly, the cartridge electrophoresis device, is that it is suitable for mass-production which provides efficiency in point-of-care technologies. The diagnostic system can provide a low cost screen test for monitoring hemoglobin levels in a subject. It is mobile and easy-to-use; it can be performed by anyone after a short (30 minute) training. The diagnostic system described herein can integrate with a mobile device (e.g., IPhone, IPod) to produce objective and quantitative results. If necessary, cartridge electrophoresis devices and/or their components may be sterilized (e.g., by UV light) and assembled in sterile laminar flow hood. Sterile biomedical grade silicon tubing (Tygon Biopharm Plus) may be integrated to the cartridge electrophoresis devices and cartridge electrophoresis devices may be sealed to prevent any leakage. Further, tubing allows simple connection to other platforms, such as in vitro culture systems for additional analyses if needed.

In other embodiments, a mobile imaging and quantification algorithm can be integrated into the diagnostic system and/or reader. The algorithm can achieve reliable and repeatable test results for data collected in all resource settings of the diagnostic system.

Other embodiments described herein relate to a method of quantifying hemoglobin level and detecting hemoglobin variants in a blood sample. Generally, the method includes generally combining a blood sample obtained from a subject with a calibrator, which has a different electrophoretic mobility and, optionally, different color than the hemoglobin the blood sample. The combined blood sample and calibrator can then be introduced to an electrophoresis device that induces migration and separation of optically detectable bands of the calibrator and hemoglobin and/or separated hemoglobin phenotypes of the blood sample. The bands of the calibrator and hemoglobin and/or separated hemoglobin phenotypes can then be optically detected and tracked and band detection data based on the detected and tracked bands of migrated and separated calibrator and hemoglobin and/or separated hemoglobin variants can be generated. The hemoglobin level and/or hemoglobin variant phenotypes can then be determined based on the determined hemoglobin level and detected hemoglobin variants. The determined hemoglobin level and/or detected hemoglobin variants can be indicative of whether the subject has anemia.

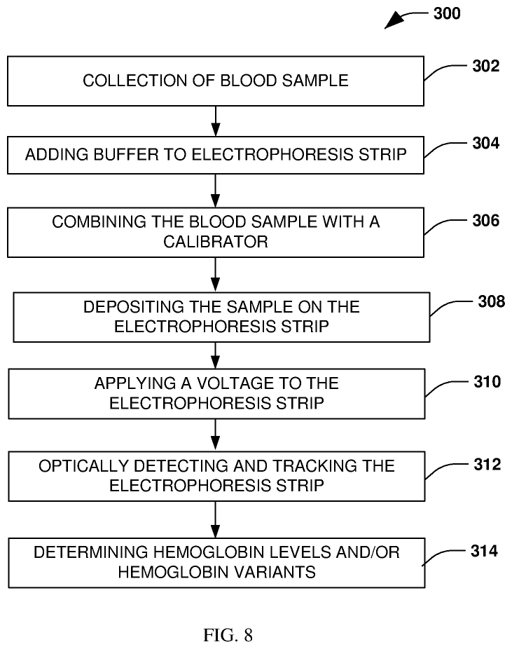

FIG. 8 illustrates an example of an analysis method of quantifying hemoglobin level and detecting hemoglobin variants in a blood sample. An initial step 302 of the method 300 can include the collection of a patient sample for analysis, in this example, a blood sample.

At 304, a buffer can be added to the electrophoresis strip in preparation for the electrophoresis testing of the collected blood sample.

In some embodiments, the buffer solution can be mildly basic, for example, a pH of about 7.5 to about 8.7, (e.g., pH 8.4). Optionally, a generally used additive may be added to the above-mentioned buffer solution. Examples thereof include surfactants, various polymers, hydrophilic low-molecular-weight compounds, and the like. By way of example, the buffer solution can include Tris/Borate/EDTA, at a pH of 8.4.

The collected blood sample 302 is then mixed at step 306, with a calibrator. The added calibrator can assist with visualizing the completed electrophoresis results. For example, a calibrator having a different electrophoretic mobility than hemoglobin or hemoglobin type at a predetermined applied voltage. The calibrator will move at a different rate relative to the hemoglobin or hemoglobin type containing portion of the blood sample across the electrophoresis strip in response to the applied voltage. The calibrator can have a color, or other optical properties different than the hemoglobin that makes visualizing the calibrator easier relative to the hemoglobin and/or hemoglobin phenotype on the electrophoresis strip.

In some embodiments, the calibrator can have a substantially different electrophoretic mobility than the hemoglobin upon application of the applied electric field to the electrophoresis strip. The electrophoretic mobility of the calibrator relative to the hemoglobin is such that calibrator band can achieve separation from the hemoglobin band in the region interest prior to completion of separation of the hemoglobin variant phenotypes.

In some embodiments, the calibrator band can achieve separation from the hemoglobin band in less than about 2.5 minutes, less than about 2 minutes, or less than about 100 seconds upon application of the applied electric field to the electrophoresis strip.

In some embodiments, the calibrator can also lyse the cells of the blood sample and have a substantially different color in the visible spectrum than the hemoglobin. In one example, the calibrator can be xylene cyanol, which has a blue color compared to a red color of the hemoglobin and/or hemoglobin phenotype.

In other embodiments, the bands of hemoglobin phenotypes are separated after the calibrator band achieves separation from the hemoglobin band and less than about 8 minutes upon application of the electric field.

It will be appreciated that the calibrator need not lyse the cells of the blood sample and that a separate lysing agent can be mixed with the blood sample to lyse the cells. Lysing agents can include fluids, such as water or various chemicals, and powders. Additionally, mechanical lysing can be used, such as by sonication, maceration and/or filtering, to achieve adequate lysing of the cells of the blood sample in preparation for analysis of the sample.

It will also be appreciated that other electrophoretic markers that have a similar or different electrophoretic mobility than the hemoglobin and/or hemoglobin phenotypes can be added to the blood sample. The other markers can have the same or a different color than hemoglobin and have molecular weights that vary between 200 g/mol to 1,000 g/mol, for example, about 300 g/mol to about 800 g/mol, or about 400 g/mol to about 700 g/mol.

At 308 the lysed and calibrated blood sample can be deposited onto the electrophoresis strip in a controlled manner, preferably applied in a “line” perpendicular to the length of the electrophoresis strip. The controlled manner of deposition can include controlling the amount of blood sample deposited, the area across which the blood sample is deposited, the shape of the area across which the blood sample is deposited and/or other deposition characteristics. One or more systems and/or components of the reader and/or cartridge can be used to deposit the blood sample in the controlled manner onto the electrophoresis strip.

With the blood sample deposited onto the electrophoresis strip, a voltage can be applied across the electrophoresis strip at 310 to cause migration of the hemoglobin and calibrator in the blood sample. The voltage or current can be applied at a predetermined level or series of levels and for an amount of time. As discussed previously, the application time of the voltage can be predetermined or based on the movement of one or more bands of the patient sample, measurement of an electrical parameter, such as resistance or an added compound/component. A higher applied voltage can cause the bands to move across the electrophoresis strip at a greater speed, however, the band shape can be distorted making the interpretation of the banding difficult. A lower applied voltage can increase band fidelity but can take a longer time to perform the requisite testing. The applied voltage can be selected to optimize testing efficiency while maintaining a desired or minimum fidelity level. Further, the applied voltage can be varied during testing, such as applying a higher voltage initially and then applying a lower voltage. The varied application of the voltage can cause the initial band separation and movement and the later applied lower voltage can assist with increasing the fidelity of the resultant banding pattern. Additionally, varying voltages and/or currents can be applied during the electrophoresis process in response to a measurement of the bands formed by the blood and/or the band or bands formed by the calibrator in a predetermined ratio, to maintain a constant rate of travel of the calibrator or a portion thereof.

The hemoglobin and calibrator will separate initially into a single hemoglobin band representing or indicative of the level or amount of hemoglobin in the sample and a calibrator band, and then the single hemoglobin band will separate into hemoglobin variant phenotype bands due to the applied voltage and the physical and electrical properties of the various hemoglobin phenotypes. The movement of a calibrator added to the patient sample can be monitored as the calibrator moves across the electrophoresis strip. That is, imaging/monitoring of the electrophoresis testing, and/or the calibrator thereon, can be performed in a continuous or timed interval manner during the testing process. For example, images of the electrophoresis process can be continuously captured, such as by a video imaging process, or the images can be captured at regular intervals based on time and/or the distance one or more bands have traveled. Once the calibrator has reached a predetermined location across the electrophoresis strip, the test can be terminated with the removal of the applied voltage.

At 312, the bands of the calibrator and hemoglobin and/or separated hemoglobin phenotypes and variants (A, F, S, and C) and can then be optically detected and tracked and band detection data based on the detected and tracked bands of migrated and separated calibrator and hemoglobin and/or separated hemoglobin variant phenotypes can be generated. In some embodiments, an electrophoresis band detection module can be configured to optically detect and track the bands of the calibrator and hemoglobin and/or separated hemoglobin variants in a region interest on the electrophoresis strip caused by the applied electric field and generate band detection data based on the detected and tracked bands of migrated and separated calibrator and hemoglobin and/or separated hemoglobin types.

Following generation of the band detection data, at 314, the hemoglobin level and/or hemoglobin variants can then be determined based on the band data of the determined hemoglobin level and detected hemoglobin phenotypes.

In some embodiments, the processor compares the intensity of the band of hemoglobin to the intensity of the band of the calibrator in the region of interest to determine hemoglobin level in the sample.

In other embodiments, the processor can compare the position of band of the calibrator relative to the positions of the separated hemoglobin phenotypes in the region of interest to determine or detect the presence of hemoglobin variants.

In other embodiments, the processor can initially determine the level of hemoglobin and then subsequently detect hemoglobin variants. The processor can also determine whether the subject has or is at risk of anemia based on the determined level of hemoglobin and/or the detected variant hemoglobin phenotypes.

The determined hemoglobin level and/or detected hemoglobin variants can be indicative of whether the subject has anemia. Hemoglobin level and hemoglobin variant phenotypes can then be determined based on the detected hemoglobin level and hemoglobin variant phenotypes. The determined hemoglobin level and/or detected hemoglobin variant can be indicative of whether the subject has anemia.

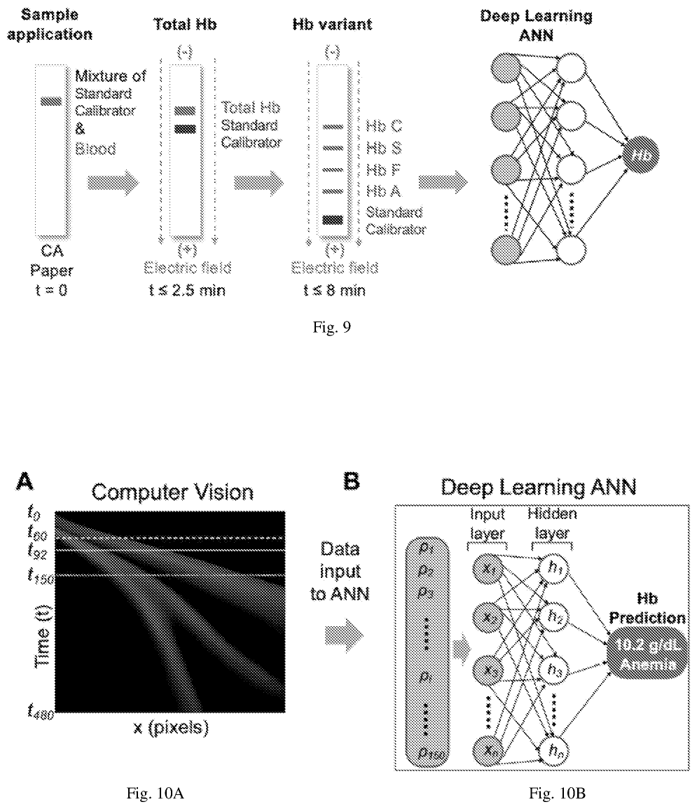

By way of example as shown in FIG. 9, initially, a drop of blood (red) is mixed with a calibrator (xylene cyanol, blue) and applied on the cellulose acetate paper in a cartridge (t=0) of an electrophoresis system. Within the first 2.5 minutes (t≤2.5 min), the total hemoglobin (red) and standard calibrator (blue) are electrophoretically separated, at which time blood hemoglobin level (g/dL) and anemia status is determined by an algorithm of a processor. Next, hemoglobin variant separation occurs (t≤8 min), which is then analyzed to determine the presence of major hemoglobin variants and types (e.g., Hb A, F, S, and C) in the blood sample. The entire electrophoresis process is tracked in real-time by computer vision and the captured data is analyzed by the deep learning artificial neural network (ANN) algorithm for integrated blood hemoglobin level prediction, anemia detection, and hemoglobin variant identification in a single test.

The following examples are for the purpose of illustration only and are not intended to limit the scope of the claims, which are appended hereto.

### Example

In this Example, we developed the first integrated point-of-care quantitative hemoglobin level, anemia detection, and Hb variant test: Hb Variant/Anemia (HbVA), by implementing a computer vision and deep learning artificial neural network (ANN) algorithm. We show the feasibility of this new diagnostic approach via testing 46 subjects, including individuals with or without anemia, and individuals with or without sickle cell disease (homozygous or heterozygous). We demonstrate that HbVA computer vision tracks the electrophoresis process real-time and the ANN algorithm reproducibly and accurately predicts blood Hb concentration with a mean absolute error of 0.55 g/dL and a bias of −0.10 g/dL (95% limits of agreement: 1.5 g/dL) according to Bland-Altman analysis. Anemia determination was achieved with 100% sensitivity and 92.3% specificity with a receiver operating characteristic area under the curve (AUC) of 0.99. Within the same test, subjects with sickle cell disease were identified with 100% sensitivity and specificity. Overall, we show that computer vision and deep learning ANN algorithms can be used to extract previously inaccessible new information from the standard Hb electrophoresis, enabling, for the first time, reproducible, accurate, and integrated blood Hb level prediction, anemia detection, and Hb variant identification in a single integrated point-of-care test.

### Methods

### Fully Integrated Microchip Electrophoresis Cartridge Allows High-Volume Manufacturing

The HemeChip cellulose acetate paper-based microchip electrophoresis system (FIG. 1) facilities, for the first time, real-time tracking and quantitative analysis of hemoglobin electrophoresis process. The HemeChip cartridge is composed of two injection molded plastic parts made of Optix® CA-41 Polymethyl Methacrylate Acrylic. This single-use, cartridge-based design was transformed from a proof-of-concept laboratory prototype to a version that supports low-cost mass-production via injecting molding. The top and bottom parts were manufactured with a 1+1 injection mold. Cartridge design embodies specific geometrical features and a precisely designed energy director for rapid ultrasonic welding after assembly. Cellulose acetate paper was chosen because of its stability over environmental conditions. Cartridge layout, plastic material selection, and injection molding process were engineered to achieve structural integrity, uniform optical clarity, and high light transmission (up to 80%) in the visible spectrum. The injection molded HemeChip cartridge embodies a pair of round corrosion-resistant, biomedical grade stainless steel 316 electrodes. HemeChip electrodes provide oxidative resistance, stability against electrochemical reactions during operation, and reliability of electrical connection with the power source. The combination of high stability cellulose acetate paper, injection molded Polymethyl Methacrylate Acrylic plastic, and corrosion-resistant biomedical grade stainless-steel electrodes result in a shelf life of at least two years. The cartridge also houses a pair of buffer pools that are in direct contact with the electrode top surface and the cellulose acetate paper strip. One corner of the HemeChip cartridge is chamfered to facilitate correct orientation during use.

### Robustness and Reproducibility of HbVA

We first tested HbVA's robustness by performing 10 repeated tests using same sample. The 10 tests were performed by 2 users with 5 tests each and compared for user variance. In addition, we tested HbVA's reproducibility over low, middle and high Hb level range. We performed reproducibility test using 3 samples at different Hb levels. Each sample was tested 3 times using both HbVA and reference standard method CBC of 6.0 g/dL (≤9.0 g/dL, low Hb level), 10.4 g/dL (within 9.1-12.0 g/dL, middle Hb level) and 14.6 g/dL (within 12.1-17.0 g/dL, high Hb level).

### Material and Reagents

1× Tris/Borate/EDTA (TBE) buffer at pH 8.4 was used as background electrolyte for electrophoresis, reconstituted from 10×TBE buffer (ThermoFisher Scientific, Waltham, Mass.). Whole blood lysing buffer was prepared using ultrapure water (ThermoFisher Scientific, Waltham, Mass.) and customized standard calibrator (a fiducial marker that is negatively charged at pH 8.4) and was used to perform whole blood lysing to release Hb from red blood cells.

### HbVA Test for Integrated Hb Level Prediction, Anemia Detection and Hb Variant Identification

The HbVA test can be performed by minimally trained personnel to produce fast, accurate, and reproducible results. The complete procedure consists of three steps: (i) chip preparation, (ii) sample preparation, (iii) Hb separation (10 minutes), followed by computer vision and deep learning ANN algorithm based data analysis. Briefly, Cellulose acetate paper within HbVA cartridge is first wetted and dried by introducing 40 μL background electrolyte. The cartridge is then positioned onto the customized stamper. Sample is prepared by diluting whole blood sample into customized standard calibrator solution at 2:1. The mixed sample is then loaded into wetted cellulose acetate paper using the customized stamper toolset. Finally, 200 μL background electrolyte is injected into the buffer ports at each end of the cartridge to provide sufficient contact between electrode-electrolyte-paper to complete circuit connection and provide stable current for electrophoresis. The prepared cartridge is then housed into the HbVA reader with preset voltage applied to initiate Hb separation.

HbVA integrated tests involve 2 steps separation in progression with time. The step 1 separation takes place within 3 minutes after initiation of separation process. During the short separation time, step 1 separates total Hb (red) from lysed blood from the applied standard calibrator (blue) (FIG. 9, middle-left&10A) due to the major mass-to-charge ratio between Hb and standard calibrator. Region of interest (ROI) is defined by computer vision. Within the ROI, the relative intensity ratio of the total Hb band intensity over total standard calibrator band intensity is evaluated by ρᵢ (ρ₉2=0.39 for demonstrated frame in FIG. 10B at t=92 s). The electrophoresis separation process within ROI is tracked for the first 150 frames (FIG. 2C) and the relative intensity ratio ρᵢ is then calculated for the first 150 frames after HbVA test initiation (FIG. 10D) tracking the relative Hb band intensity to generate a time series vector ρ(t) (FIG. 10E). The obtained ρ(t) is then input to a trained artificial neural network (ANN) which performs regression by examining underlying time series data to predict the Hb level. The step 2 separation takes place between 3-10 minutes during HbVA process (FIG. 10F). As separation time progresses, step 2 separates total Hb into individual Hb subtypes according to their finer mass-to-charge ration among various Hb subtypes. After 10 minutes, the test is stopped automatically by the reader software.

### Clinical Verification of HbVA

Clinical study design, study participants, sample size calculation, and details on test methods are designed according to the Reference and Selected Procedures for the Quantitative Determination of Hb in Blood; Approved Standard—Third Edition (H15-A3) published by the CLSI. We tested 46 samples including 37 clinical patient samples and 9 healthy subject samples. Since the focus of HbVA is to perform integrated anemia detection (reflected by low Hb level) and Hb variant identification, we mainly involved samples from SCD patients, whom were known to be plausible of suffering from anemia and lower Hb levels. Among the 46 patients, 17 tested samples (37% of the total tested samples) are within Hb level range of <9.0 g/dL; 19 tested samples (41% of the total tested samples) are within Hb level range of 9.1-12.0 g/dL; and 11 tested samples (22% of the total tested samples) are within Hb level range of 12.1-17.0 g/dL. Institutional Review Board (IRB) approved study protocols included the following common objectives: to validate HBVA technology as a point-of-care platform for Hb testing, to compare the screening results obtained from HBVA with that obtained from laboratory CBC and HPLC as the standard reference methods, to determine the diagnostic accuracy of the test including sensitivity and the specificity, and to determine the feasibility of using HBVA as a point-of-care testing platform in low and middle income countries. We obtained approvals from Institutional Review Boards at University Hospitals Cleveland Medical Center (UHCMC IRB #04-17-15).

### Statistical Methods

Statistical significance (p<0.05) was determined via two-tailed Student's t-test assuming unequal variance. All statistical tests (calculation of regression correlation coefficients and Student's t-tests were conducted using Minitab 17 Statistical Software. 95% confidence intervals for sensitivity and specificity are calculated according to the efficient-score method. The Bland-Altman method was used to determine the repeatability of HbVA Hb prediction using residual analysis by comparison to the standard reference method. The coefficient of repeatability was set as 1.96 times the standard deviations of the differences between the two measurements. Sensitivity was determined as the ratio of true positive results divided by the summation of true positive results and false negative results. Specificity was determined as the ratio of true negative results divided by the summation of true negative results and false positive results.

### Deep Learning Artificial Neural Network Algorithm Development

The computer vision and deep learning processing pipeline was established using the open source Keras machine learning library on top of a TensorFlow backend. The workflow can be deconstructed into the following steps, also illustrated for a representative HBVA test in FIG. 10. Region of interest (ROI), is carefully defined to capture all relevant pixel information for both Hb band (red) and standard calibrator band (blue). Each analyzed frame is sequentially split into its constituent red and blue channels. 2) The spatial summed relative intensity between red channel and blue channels (ρᵢ=(Σ_{Y} Σ_{X})_{Red}/(Σ_{Y} Σ_{X})_{Blue}, FIG. 10E) is calculated for each frame from time 0 to 150 s, monitoring the relative abundancy of total hemoglobin and standard calibrator within that time period, to construct a relative intensity time series vector ρ(t) (FIG. 10F). ρ(t) is fed as the input feature vector to a trained artificial neural network (ANN) that examines the intensity ratio pattern and reports the corresponding Hb level in g/dL (FIG. 10B). The result is used to determine the anemia status of the test subject according to the standard.

The ANN in our workflow has been developed for a Python environment. The processing pipeline was set up using the open source Keras machine learning library on top of a TensorFlow backend. For choice of ANN performing this regression problem, we used a vanilla feed forward network, also known as a multi-layer perceptron or MLP. The constructed ANN has three densely connected layers—an input layer, a hidden dense layer, and an output layer. The input and hidden layers each have 32 nodes with rectified linear unit (ReLU) activations. The ANN takes the pre-processed relative intensity ratio time series vector ρ(t) as input feature vector. For the size of our choice of input vector ρ(t), this corresponds to 6977 trainable parameters in the network. The network was trained and tested on a comprehensive data set of 68 HbVA tests. The training set consisted of 27 samples, out of which 4 were further split into a validation set (i.e., 15% of training set). The remaining 41 samples were kept aside for the test set, and later augmented by a further set of 5 samples to make up a combined test set of 46 samples. Training was run on an NVIDIA GeForce RTX™ 2060 GPU. Assuming the error in the input data CBC responses to be normally distributed, we chose the mean squared logarithmic error (MSLE) as our choice of loss function to minimize over the training process. To prevent overfitting—along with allocating 15% of our training set into a holdout validation set, we stopped training when the validation loss performance stopped changing over a set number of epochs. The optimal network reached an MSLE loss of 0.9% for training and 1.1% for validation. Results and validation metrics for the optimal network, along with details of efficacy testing of our predictor ANN pipeline, are presented in the results section.

### Results

### HbVA Performs 2-Steps Electrophoresis in One Single Test Tracked by Real-Time Computer Vision

The fundamental principle behind the HbVA technology is Hb electrophoresis, in which different (bio)molecules including total hemoglobin, standard calibrator, and hemoglobin variants can be separated based on their charge-to-mass ratio when exposed to an electric field in the presence of a carrier substrate. HbVA is single-use and cartridge-based, which can be mass-produced at low-cost. The HbVA test works with a standard finger-prick or a heel-prick blood sample that is collected according to the World Health Organization (WHO) guidelines for drawing blood, which typically yields about 25 μL per drop. The sampled blood is mixed and lysed with standard calibrator solution. A customized sample stamper set including sample and stamper stand is used to transfer the mixture containing lysed blood and standard calibrator into the cartridge for electrophoresis. Tris/Borate/EDTA (TBE) buffer is used to provide the necessary ions for electrical conductivity at a pH of 8.4 in the cellulose acetate paper. The pH induced net negative charges of the hemoglobins and the standard calibrator molecules cause them to travel from the negative to the positive electrode when placed in an electric field (FIG. 9). HbVA performs a 2-step separation based on processing time (FIGS. 9 & 10A): during the first step, the major mobility difference between total Hb and standard calibrator allows the total Hb including all Hb variants to separates from the standard calibrator within a short period of time (<2.5 minutes) (FIG. 9, middle-left & FIG. 10A to t₀ t₁₅₀). During the second step, the finer mobility differences among Hb variants allow Hb variants to separate (FIG. 9, middle-right & FIGS. 10A t₁₅₀ to t₄₈₀ & C, inset). This feature of naturally red, visible hemoglobin, and naturally blue, visible standard calibrator (Xylene Cyanol), combined with optically clear HbVA cartridge in transmission computer vision mode within the reader's imaging chamber, negates the need for picrosirius staining, which is typically utilized in benchtop cellulose acetate hemoglobin electrophoresis.

### Deep Learning ANN Enables Integrated Hb Level Prediction, Anemia Detection, and Hb Variant Identification in Single HbVA Test

The HbVA 2-step electrophoresis separation process is tracked in real-time by computer vision (FIG. 10A-C), and analyzed using a two-step process. Briefly, for the first step (time t₀→t₁₅₀), computer vision automatically identifies band position and defines a region of interest (ROI, FIG. 10A Inset) specific for each test and tracks the band movement within the ROI (FIG. 10A). Computer vision then processes the band information and generates a time series vector ρ(t), evaluating the relative intensity between Hb band and the standard calibrator band, as input for the trained ANN (FIG. 10B). The trained ANN performs pattern recognition and regression analysis by examining underlying input data using the time series vector ρ(t) and predicts the Hb level in g/dL (FIG. 10B) as well as detects anemia over broad Hb level range (FIG. 11A-L). This is followed by the second step of Hb variant identification. Using late time (t₁₅₀→t₄₈₀) results from the same test, Hb variants are identified using previously published algorithm (FIG. 10C, FIG. 11M-T). More specifically, the time series vector ρ(t) encoding the relative intensity, ρᵢ, obtained using the information within ROI during the first 150 s in HbVA test (FIG. 10D), is evaluated as the relative intensity ratio of the total Hb band intensity over total standard calibrator band intensity for each frame (FIG. 10E&F). The trained ANN analyzes and recognizes the underlying pattern of the relative Hb intensity ρᵢ and associates this pattern with a hemoglobin level and corresponding anemia status. Within the same test, the HbVA algorithm then tracks Hb variant band migration and identifies Hb variants based on their final locations at the end of test (FIG. 10C, t≤8 min). In case of a single Hb variant detected, such as Hb SS (FIG. 11M&Q) or Hb AA (FIG. 3P&T), the HbVA algorithm reports a percentage value greater of >90%, which agrees with the results reported by the reference standard method (HPLC). If there is more than one peak identified, then the areas under each of the peaks are calculated and the relative percentages are reported, for example in the cases of Hb AS (FIG. 11N&R), Hb SC (FIG. 11O&S). The HbVA Hb variant identification feature has been previously extensively tested and validated. In this manuscript, we emphasis on verification of the new feature of Hb level prediction and anemia detection enabled by the new information, the relative Hb intensity pattern, extracted from the standard electrophoresis using computer vision and deep learning ANN methods.

### Efficacy of ANN Based Processing Pipeline

We designed and implemented a data analyses pipeline that employs a deep learning artificial neural network (ANN) performing regression on the HbVA image data predicting Hb level and detecting anemia. Design and implementation details of the pipeline can be found in methods section. The efficacy of our network's performance has been validated using a standard repeated sub sampling validation routine. Details and results have been quoted in the Supplementary Information. The final optimally trained network reached 93.8% accuracy on a final test set of 46 samples.

### HbVA Tests are Robust and Agnostic to User Variance

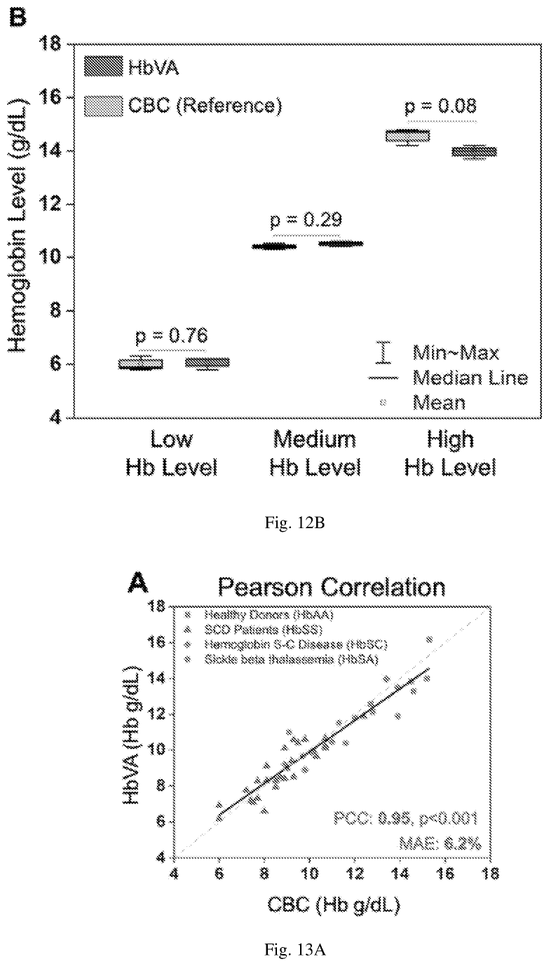

HbVA utilizes a blue standard calibrator with consistent charge-to-mass ratio at the test pH of 8.4. Utilization of relative Hb intensity over standard calibrator intensity as ANN input for Hb level prediction compensates for user variance and enhances test robustness (FIG. 12A, user 1 vs user 2). Among the 10 repeated tests (FIG. 12A), HbVA predicted consistent Hb levels at variance within ±0.6 g/dL (<4.7%). No significant difference (p=0.29) is observed on the measured Hb level between user 1 (FIG. 12A left, n=5, filled red box) and user 2 (FIG. 12A right, n=5, open red box). These results indicate that HbVA predicts Hb level and detects anemia robustly and are agnostic to user variance.

### HbVA Reproducibly Predicts Hb Level for Anemia Detection

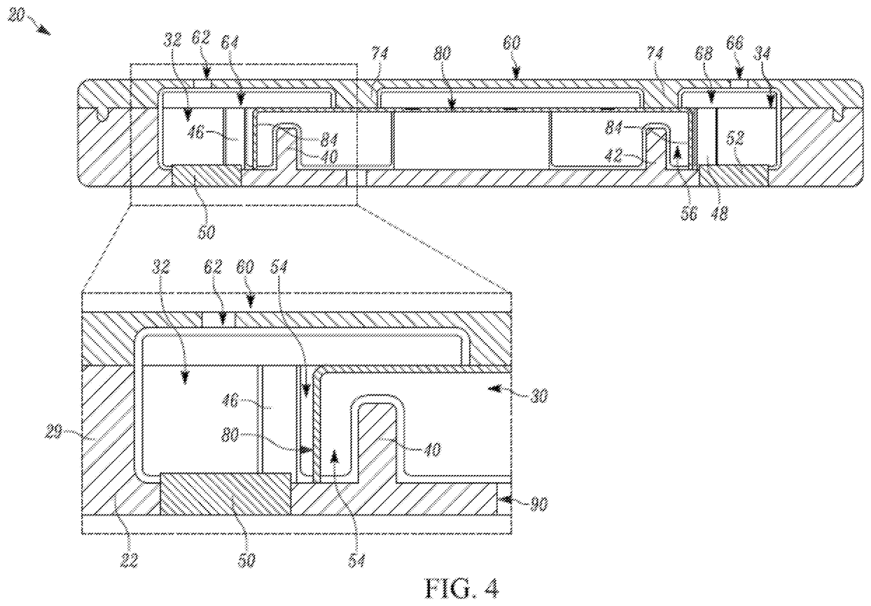

Both clinical measurement and POC measurement of Hb level have inherent variability. We performed reproducibility test using 3 samples at different Hb ranges. Each sample was tested 3 times using both HbVA and reference standard method CBC of 6.0 g/dL (≤9.0 g/dL, low Hb level), 10.4 g/dL (within 9.1-12.0 g/dL, middle Hb level) and 14.6 g/dL (within 12.1-17.0 g/dL, high Hb level). HbVA predicted Hb level consistently over all 3 tested samples (FIG. 12B). The standard deviation among individual test were determined to be ±0.2 g/dL, ±0.1 g/dL and ±0.3 g/dL (CV %=3.8%, 1.0% and 1.8%, respectively) for samples at Hb level of 6.0 g/dL (≤9.0 g/dL, low Hb level), 10.4 g/dL (within 9.1-12.0 g/dL, middle Hb level) and 14.6 g/dL (within 12.1-17.0 g/dL, high Hb level), respectively (mean Hb level reported from CBC) (FIG. 4B). In addition, HbVA predicted Hb levels are within ±0.6 g/dL when compared with CBC results demonstrating consistent agreement with the reference standard over the inspected Hb level range. These results indicate HbVA predicts Hb levels reproducibly over low, middle and high Hb levels.

### HbVA Predicts Hb Level Accurately

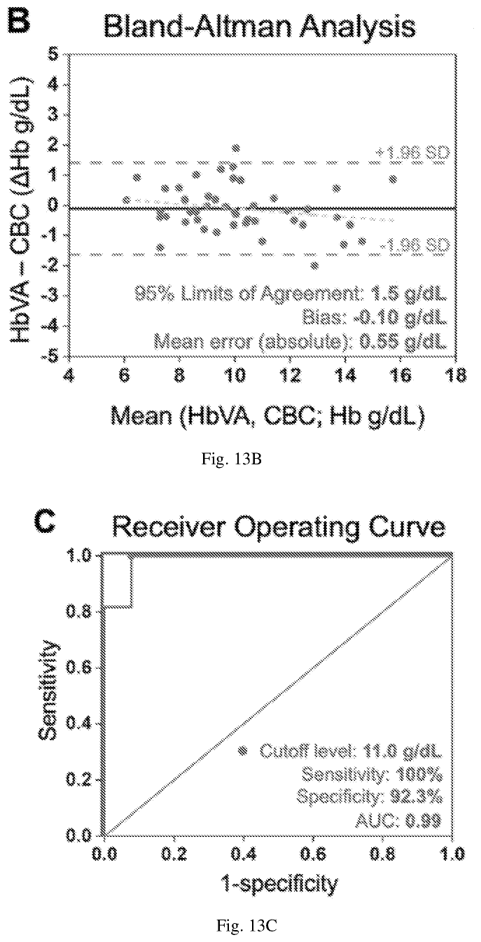

The scattered plot includes the ANN predicted Hb levels by HbVA (y axis) versus the Hb levels reported by the standard reference (CBC) within 46 tested samples with a variety of Hb variants mixed with healthy subjects (FIG. 13A). The scattered plot demonstrates Pearson correlation coefficient (PCC) of 0.95, p<0.001 revealing strong association between HbVA predicted Hb levels and CBC reported Hb levels (FIG. 13A). Bland-Altman analysis showed HbVA predicts blood Hb levels with a mean absolute error of 0.55 g/dL and a bias of −0.10 g/dL (95% limits of agreement: 1.5 g/dL) in 46 patients (FIG. 13B). The receiver-operating characteristic analysis revealed that HbVA achieves a strong performance with an area under the curve of 0.99 (FIG. 13C) and highlights the accuracy of HbVA through the entire range of tested Hb levels (6.0-15.3 g/dL). Hb levels and HbVA measured residual (r=−0.07) indicate that HBVA performance remained consistent throughout range of tested Hb level (FIG. 13B), indicating the potential for HBVA's new indication as integrated screening tool for anemia. In fact, this degree of accuracy is on par with reported accuracy values in POC settings of other clinically used single-function anemia detection devices.

HbVA Performs Integrated Anemia Detection and Hb Variant Identification with High Sensitivity and Specificity

We investigated the sensitivity and specificity of HbVA anemia detection from ANN predicted Hb levels, using single Hb cutoff level of 11.0 g/dL to differentiate anemia and non-anemia subjects. Hemoglobin level<11.0 g/dL is a well-established Hb level threshold. The sensitivity and specificity of HBVA to detect anemia was 100% and 92.3% (95% CI, 84.6%-100%), respectively (Table 1). The high sensitivity and specificity indicate the potential for this test to serve as integrated tool for anemia. We investigated sensitivity and specificity of HbVA Hb variant identification by comparing HbVA identified Hb variant with standard reference method HPLC reported results. Overall, HBVA demonstrated 100% sensitivity and specificity on identifying Hb variants including HbSS, HbAS, HbSC and HbAA among the tested 46 samples using the results from the same tests for Hb level measurement and anemia detection (Table 2).

TABLE 1
Sensitivity and specificity of Hb Variant/Anemia
on determining anemia severity
Anemia vs. Non-Anemia (Cut-off: 11.0 g/dL)

| | |
|---|---|
| True Positive, TP | 33 |
| True Negative, TN | 12 |
| False Positive, FP | 1 |
| False Negative, FN | 0 |
| Sensitivity, | 100% |
| TP/(TP + FN) | |
| Specificity, | 92.30% |
| TN/(TN + FP) | |

TABLE 2
Sensitivity and specificity of Hb Variant/Anemia
on Hb variant identification

| | SCD-SS vs. | SCD-SC vs. | Normal vs. |
|---|---|---|---|
| | others | others | others |
| True Positive, TP | 9 | 6 | 7 |
| True Negative, TN | 37 | 40 | 39 |
| False Positive, FP | 0 | 0 | 0 |
| False Negative, FN | 0 | 0 | 0 |
| Sensitivity, | 100% | 100% | 100% |
| TP/(TP + FN) | | | |
| Specificity, | 100% | 100% | 100% |
| TN/(TN + FP) | | | |

### Diagnosis Profile for HbVA Integrated Hb Level Prediction, Anemia Detection and Hb Variant Identification

FIG. 5A concludes the diagnosis profile reported by ANN using HBVA test results on all 46 with various Hb variants. Overall, 7 healthy subjects, 6 sickle Hb C (HbSC) patients, 5 sickle beta thalassemia (HbSA) patients, and 28 SCD (HbSS) patients were tested. It needs to be noticed that 21 of the 28 SCD patients tested in this study have been taking transfusion therapies thus their Hb variants were identified as HbAS or HbSA by both HbVA and standard reference method HPLC. Among the 46 tested patients, HbVA demonstrated high accuracy in Hb level prediction as well as high sensitivity and specificity in both anemia detection as well as Hb variant identification, comparing with the standard reference methods CBC and HPLC.

From the above description of the invention, those skilled in the art will perceive improvements, changes and modifications. Such improvements, changes, and modifications are within the skill of the art and are intended to be covered by the appended claims. All patents and publications identified herein are incorporated by reference in their entirety.

Having described the invention, the following is claimed:

1. A diagnostic system for detecting hemoglobin, comprising:
a cartridge that includes an electrophoresis strip structured to receive a hemolysate of a blood sample that is combined with a calibrator, which has a different electrophoretic mobility than hemoglobin in the blood sample; and first and second electrodes configured to generate an electric field across the electrophoresis strip; wherein the application of an electric field to the first and second electrodes induces migration and separation of bands of the calibrator and hemoglobin and/or separated hemoglobin phenotypes in the hemolysate delivered to the electrophoresis strip; and
an electrophoresis band detection module configured to optically detect and track the bands of the calibrator and hemoglobin and/or separated hemoglobin phenotypes in a region of interest on the electrophoresis strip caused by the applied electric field and to generate band detection data based on the detected and tracked bands of migrated and separated calibrator and hemoglobin and/or separated hemoglobin phenotypes; and
a processor that receives and analyzes the band detection data to determine hemoglobin level and hemoglobin variants and generate diagnostic results based on the hemoglobin level and hemoglobin variants.

2. The diagnostic system of claim 1, wherein the processor compares the intensity of the band of hemoglobin to the intensity of band of the calibrator in the region of interest to determine hemoglobin level in the sample.

3. The diagnostic system of claim 1, wherein the processor determines initially the level of hemoglobin and then the presence of hemoglobin variants.

4. The diagnostic system of claim 1, wherein the calibrator lyses blood cells when mixed with the blood sample.

5. The diagnostic system of claim 1, wherein the electrophoretic mobility of the calibrator relative to the hemoglobin is such that calibrator band achieves separation from the hemoglobin band in the region interest and prior to completion of separation of hemoglobin phenotypes.

6. The diagnostic system of claim 1, wherein the calibrator band achieves separation from the hemoglobin band in less than about 2.5 minutes.

7. The diagnostic system of claim 1, wherein the calibrator has a substantially different color than the hemoglobin on the electrophoresis strip.

8. The diagnostic system of claim 1, wherein the calibrator comprises xylene cyanol.

9. The diagnostic system of claim 1, wherein the hemolysate of the blood sample introduced into the sample loading port is less than 10 μL.

10. The diagnostic system of claim 1, wherein the electrophoresis strip is saturated with a tris/borate/EDTA buffer solution.

11. The diagnostic system of claim 1, wherein the processor is configured to diagnose whether the subject has or is at risk of anemia.

12. A method of detecting anemia in a subject in need thereof, the method comprising:
combining a blood sample from the subject with a calibrator, which has a different electrophoretic mobility than hemoglobin in the blood sample,
introducing the combined blood sample and calibrator to an electrophoresis device that induces migration and separation of optically detectable bands of the calibrator and hemoglobin and/or separated hemoglobin phenotypes of the blood sample introduced to the electrophoresis strip upon application of an electric field; and
optically detecting and tracking with an electrophoresis band detection module the bands of the calibrator and hemoglobin and/or separated hemoglobin phenotype types in a region of interest and generating band detection data based on the detected and tracked bands of migrated and separated calibrator and hemoglobin and/or separated hemoglobin phenotypes; and
determining hemoglobin level and detected hemoglobin variants based on the determined hemoglobin level and detected hemoglobin phenotypes, wherein determined hemoglobin level and/or detected hemoglobin variants is indicative of whether the subject has anemia.

13. The method of claim 12, comparing the intensity of the band of hemoglobin to the intensity of the band of the calibrator in the region of interest to determine hemoglobin level.

14. The method of claim 12, wherein the level of hemoglobin is determined before detecting hemoglobin phenotypes.

15. The method of claim 12, wherein the calibrator lyses the cells of the blood sample.

16. The method of claim 12, wherein the electrophoretic mobility of the calibrator relative to the hemoglobin is such that calibrator band achieve separation from the hemoglobin band in the region interest and prior to completion of separation of hemoglobin phenotypes.

17. The method of claim 12, wherein the calibrator band achieves separation from the hemoglobin band in less than about 2.5 minutes upon application of the electric field.

18. The method of claim 12, wherein the hemoglobin variants are separated after the calibrator band achieves separation from the hemoglobin band and less than about 8 minutes upon application of the electric field

19. The method of claim 12, wherein the calibrator has a substantially different color than the hemoglobin on the electrophoresis strip.

20. The method of claim 12, wherein the calibrator comprises xylene cyanol.

21. The method of claim 12, wherein the buffer solution comprises tris/borate/EDTA buffer solution.
